(19) United States
(12) Patent Application Publication
(10) Pub. No.: US 2025/0179582 A1
(43) Pub. Date: Jun. 5, 2025

(54) METHODS FOR TREATING CANCER PATIENTS WITH HOMOLOGOUS RECOMBINATION DEFICIENCY BASED ON TEMPLATED INSERTIONS

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Daniel S. Higginson, New York, NY (US); Grace M. Moore, New York, NY (US)

(21) Appl. No.: 18/840,416

(22) PCT Filed: Feb. 21, 2023
(86) PCT No.: PCT/US2023/062957

§ 371 Date: Aug. 21, 2024

Related U.S. Application Data
(60) Provisional application No. 63/312,568, filed on Feb. 22, 2022.

(51) Int. Cl. C12Q 1/6886; A61K 31/166; A61K 31/282; A61K 31/4184; A61K 31/454; A61K 31/502; A61K 31/5025; A61K 31/506; A61K 31/55; A61K 33/243; A61P 35/00

(52) U.S. Cl. CPC C12Q 1/6886; A61K 31/166; A61K 31/282; A61K 31/4184; A61K 31/454; A61K 31/502; A61K 31/5025; A61K 31/506; A61K 31/55; A61K 33/243; A61P 35/00; C12Q 2600/106; C12Q 2600/156

(57) ABSTRACT

The present disclosure provides methods for determining whether a cancer patient with homologous recombination deficiency will benefit from treatment with PARP inhibitors or platinum agents. These methods are based on screening a cancer patient for the presence of templated insertions (TINS), including direct-repeat templated insertions (drTINS) and inverted templated insertions (iTINS).

### CROSS-REFERENCE TO RELATED APPLICATIONS

Ths application claims the benefit of and priority to U.S. Provisional Patent Application No. 63/312,568, filed Feb. 22, 2022, the contents of which are incorporated herein by reference in its entirety.

### STATEMENT OF GOVERNMENT SUPPORT

This invention was made with government support under CA08748 awarded by the National Institutes of Health. The government has certain rights in the invention.

### TECHNICAL FIELD

The present technology relates to methods for determining whether a cancer patient with homologous recombination deficiency (HRD) will benefit from treatment with PARP inhibitors or platinum agents. These methods are based on screening a cancer patient for the presence of templated insertions (TINS), including direct-repeat templated insertions (drTINS) and inverted templated insertions (iTINS).

### BACKGROUND

The following description of the background of the present technology is provided simply as an aid in understanding the present technology and is not admitted to describe or constitute prior art to the present technology.

Cancer cells defective in homologous recombination (HR) are responsive to DNA crosslinking chemotherapies, PARP inhibitors, and inhibitors of polymerase theta, a key mediator of the backup pathway alternative end-joining. Such cancers include those with pathogenic bi-allelic alterations in core HR genes and another cohort of cases that exhibit sensitivity to the same agents and harbor genomic hallmarks of HR deficiency (HRD). In advanced epithelial ovarian cancers, the standard of care currently consists of optimal cytoreductive surgery followed by platinum-based chemotherapy (www.nccn.org/professionals/physician_gls/pdf/ovarian.pdf). However, nearly all stage III and all stage IV cancers recur, and thus overall survival following initial surgery and platinum-based chemotherapy is considered highly determined by inherent platinum sensitivity (Markman, M. et al., J Clin Oncol 22, 3120-3125 (2004); Bouberhan, S. et al., J Clin Oncol 37, 2424-2436 (2019)).

Accordingly, there is an urgent need for developing reliable and accurate methods for predicting whether a HRD cancer patient will benefit from treatment with PARP inhibitors or platinum agents.

### SUMMARY OF THE PRESENT TECHNOLOGY

In one aspect, the present disclosure provides a method for selecting a cancer patient for treatment with a PARP inhibitor or a platinum agent comprising: (a) detecting levels of templated insertions (TINS) in a biological sample obtained from the cancer patient that are elevated relative to a control sample obtained from a healthy subject or a predetermined threshold; and (b) administering to the cancer patient an effective amount of a PARP inhibitor or a platinum agent. In another aspect, the present disclosure provides a method for prolonging survival of a cancer patient comprising administering to the cancer patient an effective amount of a PARP inhibitor or a platinum agent, wherein levels of templated insertions (TINS) in a biological sample obtained from the cancer patient are elevated relative to a control sample obtained from a healthy subject or a predetermined threshold. In some embodiments of the methods disclosed herein, the levels of TINS are detected via next-generation sequencing.

In any of the preceding embodiments of the methods disclosed herein, the TINS comprise direct-repeat TINS (drTINS) and/or inverted TINS (iTINS). Additionally or alternatively, in some embodiments of the methods disclosed herein, the TINS (e.g., drTINS and/or iTINS) have a length ranging from 5 base pairs (bps) to 20 base pairs (bps) or 20 base pairs (bps) to 50 base pairs (bps). In certain embodiments, the length of the TINS (e.g., drTINS and/or iTINS) is 5 bps, 6 bps, 7 bps, 8 bps, 9 bps, 10 bps, 11 bps, 12 bps, 13 bps, 14 bps, 15 bps, 16 bps, 17 bps, 18 bps, 19 bps, 20 bps, 21 bps, 22 bps, 23 bps, 24 bps, 25 bps, 26 bps, 27 bps, 28 bps, 29 bps, 30 bps, 31 bps, 32 bps, 33 bps, 34 bps, 35 bps, 36 bps, 37 bps, 38 bps, 39 bps, 40 bps, 41 bps, 42 bps, 43 bps, 44 bps, 45 bps, 46 bps, 47 bps, 48 bps, or 49 bps.

Additionally or alternatively, in some embodiments, the cancer patient is diagnosed with, or is at risk of having a homologous recombination deficiency (HRD). In certain embodiments, the HRD comprises mutations that result in the inactivation of at least one of the following genes: PALP2/FANCN, BRIP1/FANCJ, BARD1, RAD51, RAD51 paralogs (RAD51B, RAD51C, RAD51D, XRCC2, XRCC3), FANCA, FANCB, FANCC, FANCD2, FANCE, FANCG, FANCI, FANCL, FANCM, FAN1, SLX4/FANCP and ERCC1. In any and all embodiments of the methods disclosed herein, the cancer patient harbors a mutation in one or more driver mutations selected from among BRCA2, BRCA1, IDH1, CDK4 and RB1. Additionally or alternatively, in some embodiments, the cancer patient is suffering from a cancer selected from among breast cancer, ovarian cancer, prostate cancer, and pancreatic cancer. In certain embodiments, the cancer patient exhibits stage I, stage II, stage III or stage IV cancer. Additionally or alternatively, in some embodiments, the cancer patient has not received at least one prior line of anti-cancer therapy. In other embodiments, the cancer patient has received at least one prior line of anti-cancer therapy. Examples of anti-cancer therapy include chemotherapy, radiation therapy, surgery or any combination thereof.

In any and all embodiments of the methods disclosed herein, the PARP inhibitor is a PARP-specific inhibitory nucleic acid (e.g., sgRNAs, antisense RNAs, ribozymes, or shRNAs), iniparib, olaparib, niraparib, rucaparib, talazoparib, veliparib, AG014699, CEP 9722, MK 4827, BMN-673, E7016, 3-aminobenzamide, or an anti-PARP neutralizing antibody.

In any and all embodiments of the methods disclosed herein, the platinum agent is cisplatin, carboplatin, oxaliplatin, nedaplatin, triplatin tetranitrate, phenanthriplatin, pyriplatin, picoplatin, or satraplatin.

Additionally or alternatively, in some embodiments of the methods disclosed herein, the biological sample obtained from the cancer patient comprises biopsied tumor tissue, whole blood, plasma, or serum.

Additionally or alternatively, in some embodiments, the methods of the present technology further comprise determining an additional HRD signature in the cancer patient. In some embodiments, the additional HRD signature comprises one or more of loss of heterozygosity (LOH), large scale state transitions (LST), and telomeric imbalance (tAI). In certain embodiments, the additional HRD signature comprises one or more of single base substitution signature 3 (SBS3), single base substitution signature 8 (SBS8), short tandem duplications (RefSig R3), short deletions (RefSig R5), microhomology-mediated indels, and HRD score. In other embodiments, the additional HRD signature comprises ID8 signature, and ID6 signature.

### BRIEF DESCRIPTION OF THE DRAWINGS

FIG. 1A shows a diagram of current, known mutational signatures, including base substitutions, large rearrangements, structural variants, small indels, and composite homologous recombination deficiency (HRD) scores.

FIG. 1B shows the relationship between the presence of a driver and the HRD signature through a pipeline of data analysis from PCAWG WGS data to univariate and multivariate analysis of signatures and ovarian cancer survival data.

FIG. 1C shows LEFT: Heatmap of driver genes with significant associations to two or more signatures from the multivariate regression model. Only showing drivers with available survival data from either PCAWG or TCGA ovarian cancer data. Significance of the association is shown by the size of the dot. Color indicates the contribution to the model. Red is a positive coefficient and blue is negative. RIGHT: Cox regression hazard plots of stage III and IV ovarian cancer cases filtered for treatment when available from PCAWG and TCGA.

FIG. 1D shows Kaplan-Meier curves for stage III and IV ovarian cancer cases from PCAWG for HRDetect, ID6, and ID8, shown with and without pathogenic, biallelic BRCA mutations. Groups are divided by known thresholds. Signature thresholds are defined as 0.7 or greater for HRDetect and greater than 0 for ID6 and ID8. P-values from log-rank test.

FIG. 2A shows the schematics of the small deletions that often result from polymerase theta (Pol θ) repairing stranded double-strand breaks.

FIG. 2B compares deletions in the 5-30 bp range between pathogenic, biallelically mutated BRCA1, BRCA2 samples and wild-type PCAWG samples.

FIG. 2C compares pathogenic, biallelic mutated BRCA1, BRCA2, and WT MH lengths as an average proportion of all indels in PCAWG data.

FIG. 2D shows a heatmap of driver gene significantly associated with a TMEJ signature. Significance of the association is shown by the size of the dot. Color indicates the contribution to the model. Red is a positive coefficient and blue is negative.

FIG. 2E shows cox regression hazard plots for PCAWG stage III and IV ovarian cancer samples for each of the TMEJ signatures, shown with and without pathogenic, biallelic BRCA mutations. Groups are divided by known and computed thresholds. Signature thresholds were defined as greater than 0 for ID6 and ID8 and as the median value of TMEJ signatures in PCAWG stage III and IV ovarian cancer cases.

FIG. 2F shows Kaplan-Meier curves for TMEJ4 in PCAWG stage III and IV ovarian cancer data, shown with and without pathogenic, biallelic BRCA mutations. Groups are divided by computed thresholds. Signature thresholds were defined as the median value of TMEJ signatures in PCAWG stage III and IV ovarian cancer cases. P-values from log-rank test.

FIG. 2G TMEJ4 proportions in pathogenic, biallelic mutated BRCA1, BRCA2, and WT PCAWG breast, prostate, ovarian, and pancreas samples. P-values from Mann-Whitney U test.

FIG. 3A shows a diagram of inverted template insertions (iTINS) foldback insertion mechanism.

FIG. 3B shows a diagram of direct repeat insertions (drTINS) direct insertion mechanism.

FIG. 3C shows the location of template sequence relative to insertion site.

FIG. 3D shows the distribution of TINS size.

FIG. 3E shows the heatmap of driver genes significantly associated with at least one TINS signature. Significance of the association is shown by the size of the dot. Color indicates the contribution to the model. Red is a positive coefficient and blue is negative.

FIG. 3F shows TINS, iTINS, and drTINS proportions in pathogenic, biallelic mutated BRCA1, BRCA2, and WT PCAWG breast, prostate, ovarian, and pancreas samples. P-values from Mann-Whitney U test.

FIG. 3G shows Cox regression hazard plots for all PCAWG stage III and IV ovarian cancer samples, shown with and without pathogenic, biallelic BRCA mutations. Groups are divided by computed thresholds. Signature thresholds are defined as the median value for TINS signatures in PCAWG stage III and IV ovarian cancer cases.

FIG. 3H shows Kaplan-Meier curves for TINS, iTINS, and drTINS signatures in PCAWG stage III and IV ovarian cancer data, shown with and without pathogenic, biallelic BRCA mutations. Groups are divided by computed thresholds. Signature thresholds are defined as the median value for TINS signatures in PCAWG stage III and IV ovarian cancer cases. P-values from log-rank test.

FIG. 4A shows Cox regression hazard plots of known and novel signatures for PCAWG stage III and IV ovarian cancer cases without pathogenic, biallelic BRCA mutations. Groups are divided by known and computed thresholds. Signature thresholds are defined as 0.7 or greater for HRDetect, greater than 0 for ID6, greater than 42 for HRD, and as the median value for TMEJ4 and TINS signatures in PCAWG stage III and IV ovarian cancer cases.

FIG. 4B shows Multivariate Cox regression results for signatures with ≥0.05 p-value in survival models.

FIG. 4C shows the Oncoprint of signatures for PCAWG stage III and IV ovarian cancer cases. Groups are divided by known and computed thresholds. Signature thresholds are defined as 0.7 or greater for HRDetect and as the median value for and TINS signatures in PCAWG stage III and IV ovarian cancer cases.

FIG. 4D shows Kaplan-Meier curves of cox regression significant signatures. Groups are divided by known and computed thresholds. Signature thresholds are defined as 0.7 or greater for HRDetect, greater than 0 for ID6, and as the median value for TINS signatures in PCAWG stage III and IV ovarian cancer cases. P-values from log-rank test.

FIG. 4E shows Kaplan-Meier curves of cox regression significant signatures. Groups are divided by known and computed thresholds. Signature thresholds are defined as 0.7 or greater for HRDetect, greater than 0 for ID6, and as the median value for TINS signatures in PCAWG stage III and IV ovarian cancer cases. P-values from log-rank test.

FIG. 5A shows a heatmap of driver genes with significant associations to two or more signatures from multivariate regression model without available survival data from either PCAWG or TCGA. Significance of the association is shown by the size of the dot. Color indicates the contribution to the model. Red is a positive coefficient and blue is negative.

FIG. 5B shows Kaplan-Meier curves of stage III and IV ovarian cancer cases from PCAWG for HRD and SBS3 with and without BRCA mutations. Groups are divided by known thresholds. Signature thresholds are defined as 42 or greater for HRD and greater than 0 for SBS3.

FIG. 5C shows Kaplan-Meier curves of stage III and IV ovarian cancer cases from PCAWG for SBS3, ID6, and ID8 with and without BRCA mutations. Groups are divided by novel thresholds. Signature thresholds are defined as the median value for SBS3, ID6, and ID8 in PCAWG stage III and IV ovarian cancer cases. All p-values from log-rank tests.

FIG. 6 shows survival data from TCGA stage III and IV, platinum treated ovarian cancer cases with homologous RB1 deletions. P-value from log-rank tests.

FIG. 7A shows Kaplan-Meier curves of stage III and IV ovarian cancer cases from PCAWG for TMEJ2 and TMEJ3 with and without BRCA mutations. Groups are divided by novel thresholds. Signature thresholds are defined as the median value for TMEJ2 and TMEJ3 in PCAWG stage III and IV ovarian cancer cases.

FIG. 7B shows Cox regression hazard plots for all PCAWG stage III and IV ovarian cancer samples, shown with and without pathogenic, biallelic BRCA mutations. Signature thresholds are defined as greater than 0 for ID6, ID8, and TMEJ signatures.

FIG. 7C shows Kaplan-Meier curves of stage III and IV ovarian cancer cases from PCAWG for TMEJ signatures, shown with and without pathogenic, biallelic BRCA mutations. Groups are divided by novel thresholds. Signature thresholds are defined as greater than 0 for TMEJ signatures. All p-values from log-rank tests.

FIG. 8A shows Kaplan-Meier curves of stage III and IV ovarian cancer cases from PCAWG for TINS, iTINS, and drTINS, shown with and without pathogenic, biallelic BRCA mutations. Groups are divided by novel thresholds. Signature thresholds are defined as greater than 0 for TINS signature.

FIG. 8B shows Cox regression hazard plots for all PCAWG stage III and IV ovarian cancer samples shown with and without pathogenic, biallelic BRCA mutations. Groups are divided by novel thresholds. Signature thresholds are defined as greater than 0 for TINS signature. All p-values from log-rank tests.

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectorsfor Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

The methods of the present technology are based on the identification of novel signatures of polymerase theta mediated (TMEJ) repair. Pol θ mediates small insertions representing an initial insufficient microhomology match followed by aborted synthesis, reannealing, and repair. The resected 3′ end can also snap back and anneal to itself, followed by polymerization, dissolution, and reannealing across the break, leaving behind an inverted template insertion. These events are known as templated insertions (TINS). The present disclosure demonstrates that elevated TINS genomic signatures in advanced cancers are predictive of patient responsiveness to HRD targeted therapies such as platinum agents or PARP inhibitors. The Examples described herein demonstrate that templated insertions are specifically associated with bi-allelic BRCA2 mutated genomes in a pan-cancer analysis and overall survival in advanced stage III/IV ovarian cancer cases treated with platinum-based chemotherapy, and supports the inclusion of TINS as an additional feature to predict responses to platinum agents.

### Definitions

Unless defined otherwise, all technical and scientific terms used herein generally have the same meaning as commonly understood by one of ordinary skill in the art to which this technology belongs. As used in this specification and the appended claims, the singular forms “a” “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the terms “amplify” or “amplification” with respect to nucleic acid sequences, refer to methods that increase the representation of a population of nucleic acid sequences in a sample. Nucleic acid amplification methods are well known to the skilled artisan and include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA)(TwistDx, Cambridge, UK), transcription mediated amplification, signal mediated amplification of RNA technology, loop-mediated isothermal amplification of DNA, helicase-dependent amplification, single primer isothermal amplification, and self-sustained sequence replication (3SR), including multiplex versions or combinations thereof. Copies of a particular nucleic acid sequence generated in vitro in an amplification reaction are called “amplicons” or “amplification products.”

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

“Detecting” as used herein refers to determining the presence of a mutation or genetic alterations in a nucleic acid of interest in a sample. Detection does not require the method to provide 100% sensitivity. Analysis of nucleic acid markers can be performed using techniques known in the art including, but not limited to, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol, 3:39-42 (1992)), sequencing with mass spectrometry such as matrix-assisted laser desorption/ionization time-of-flight mass spectrometry (MALDI-TOF/MS; Fu et al., Nat. Biotechnol, 16:381-384 (1998)), and sequencing by hybridization. Chee et al., Science, 274:610-614 (1996); Drmanac et al., Science, 260:1649-1652 (1993); Drmanac et al., Nat. Biotechnol, 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the expression “DNA homologous recombination (HR) pathway” has its general meaning in the art. It refers to the cellular pathway through which Double Stranded DNA breaks (DSB) are repaired by a mechanism called Homologous Recombination. Inside mammalian cells, DNA is continuously exposed to damage arising from exogenous sources such as ionizing radiation or endogenous sources such as byproducts of cell replication. All organisms have evolved different strategies to cope with these lesions. One of the most deleterious forms of DNA damage is DSB. In mammalian cells, there are two major pathways to repair DSB: Homologous recombination (HR) and Non Homologous End Joining (NHEJ). HR is the most accurate mechanism to repair DSB because it uses an intact copy of the DNA from the sister chromatid or the homologous chromosome as a matrix to repair the break.

“Deficiency in the HR pathway” or “HRD” as used herein, refers to a condition in which the HR pathway for repairing DNA is deficient or inactivated. Exemplary proteins involved in the HR pathway can encompass, but are not limited to, inactivation of at least one of the following genes: BRCA1, BRCA2, PALP2/FANCN, BRIP1/FANCJ, BARD1, RAD51, RAD51 paralogs (RAD51B, RAD51C, RAD51D, XRCC2, XRCC3), FANCA, FANCB, FANCC, FANCD2, FANCE, FANCG, FANCI, FANCL, FANCM, FAN1, SLX4/FANCP and ERCC1. See also US20170260588.

As used herein, “HRD score” refers to the number of loss of heterozygosity (LOH) regions >15 Mb, but less than a whole chromosome in length, observed within a tumour genome.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

“Large scale state transitions” or “LST” as used herein refer to breakpoints along a genome that result in genomic DNA segments of at least 3, 4, 5, 6, 7, 8, 9, 10, 11 12, 13, 14, 15, 16, 17, 18, 19 or 20 megabases in length.

As used herein, the term “library” refers to a collection of nucleic acid sequences, e.g., a collection of nucleic acids derived from whole genomic, subgenomic fragments, cDNA, cDNA fragments, RNA, RNA fragments, or a combination thereof. In one embodiment, a portion or all of the library nucleic acid sequences comprises an adapter sequence. The adapter sequence can be located at one or both ends. The adapter sequence can be useful, e.g., for a sequencing method (e.g., an NGS method), for amplification, for reverse transcription, or for cloning into a vector.

The library can comprise a collection of nucleic acid sequences, e.g., a target nucleic acid sequence (e.g., a tumor nucleic acid sequence), a reference nucleic acid sequence, or a combination thereof. In some embodiments, the nucleic acid sequences of the library can be derived from a single subject. In other embodiments, a library can comprise nucleic acid sequences from more than one subject (e.g., 2, 3, 4, 5, 6, 7, 8, 9, 10, 20, 30 or more subjects). In some embodiments, two or more libraries from different subjects can be combined to form a library having nucleic acid sequences from more than one subject.

A “library nucleic acid sequence” refers to a nucleic acid molecule, e.g., a DNA, RNA, or a combination thereof, that is a member of a library. Typically, a library nucleic acid sequence is a DNA molecule, e.g., genomic DNA or cDNA. In some embodiments, a library nucleic acid sequence is fragmented, e.g., sheared or enzymatically prepared, genomic DNA. In certain embodiments, the library nucleic acid sequences comprise sequence from a subject and sequence not derived from the subject, e.g., adapter sequence, a primer sequence, or other sequences that allow for identification, e.g., “barcode” sequences.

“Loss of heterozygosity (LOH)” may result from several mechanisms. For example, in some embodiments, a region of one chromosome is deleted in a somatic cell. The region that remains present on the other chromosome (the other non-sex chromosome for males) is an LOH region as there is only one copy (instead of two copies) of that region present within the genome of the affected cells. The LOH region can be any length (e.g., from a length less than about 1.5 Mb up to a length equal to the entire length of the chromosome). In some embodiments, an LOH event results in a copy number reduction. In other embodiments, a region of one chromosome (one non-sex chromosome for males) in a somatic cell can be replaced with a copy of that region from the other chromosome, thereby eliminating any heterozygosity that may have been present within the replaced region. In such cases, the region that remains present on each chromosome is an LOH region and can be referred to as a copy neutral LOH region. Copy neutral LOH regions can be any length (e.g., from a length less than about 1.5 Mb up to a length equal to the entire length of the chromosome).

As used herein, “microhomology” or “NM” refers to short regions of DNA sequence homology. In some embodiments, microhomology occurs directly at a breakpoint junction of a genomic rearrangement (e.g., germline or somatic breakpoint juntion). Although definitions of breakpoint microhomology vary with respect to the length of the homologous region, it can be defined as a series of nucleotides (<70) that are identical at the junctions of the two genomic segments that contribute to the rearrangement. In other embodiments, microhomology occurs adjacent to the junction of a genomic rearrangement without any overlap.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 103, 104, 105 or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, the term “overall survival” or “OS” means the observed length of life from the start of treatment to death or the date of last contact.

As used herein the term “PARP inhibitor” refers to a compound which is capable of inhibiting the activity of the enzyme polyADP ribose polymerase (PARP), a protein that is important for repairing single-strand breaks (‘nicks’ in the DNA). If such nicks persist unrepaired until DNA is replicated (which must precede cell division), then the replication itself will cause double strand breaks to form. Drugs that inhibit PARP cause multiple double strand breaks to form in this way, and in tumors with BRCA1, BRCA2 or PALB2 mutations these double strand breaks cannot be efficiently repaired, leading to the death of the cells.

As used herein, a “sample” refers to a substance that is being assayed for the presence of a mutation or alterations in a nucleic acid of interest. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “subject”, “patient”, or “individual” can be an individual organism, a vertebrate, a mammal, or a human. In some embodiments, the subject, patient or individual is a human.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

“Templated insertions” or “TINS” as used herein refer to polymerase theta (Pol θ)-mediated insertions that are 5-49 bps having nucleic acid sequences that match an equivalent length sequence (“template”) found within a window that is 50 base pairs upstream or downstream of the insertion site using human genome build hs37d5. See Carvajal-Garcia et al., Proc Natl Acad Sci USA 117, 8476-8485 (2020). In some embodiments, the insertion and template can directly match (“direct repeat TINS”). In other embodiments, a reverse complement sequence (“inverted TINS”) of the insertion matches the template. As used herein, TINS exclude tandem repeats (i.e., insertions directly adjacent to their template, or a 0 bp distance between the insertion and template sequence). As such, the length of an individual template insertion (TIN) at an insertion point ranges from 5-49 bps, and is identical or complementary to a nucleic acid sequence that is 50 base pairs upstream or downstream of the insertion site. Inverted and direct repeat TINS may be summed to create a total TINS count. The unique categories of inverted TINS (iTINS) and direct repeat TINS (drTINS) may be tallied separately. In some embodiments, raw TINS, iTINS, and drTINS counts are normalized by the total indel sum in each case to create comparable frequency counts across samples.

### Conventional HRD Signatures

Inherited germline mutations in BRCA1 and BRCA2 are associated with an elevated risk of breast, ovary, pancreas, and prostate cancer. During early carcinogenesis, the second allele often becomes defective due to loss of heterozygosity leading to cancers with two defective copies of core components of the homologous recombination (HR) pathway. HR deficient cancers constitute a clinically important subgroup.

As carcinogenesis proceeds over many cell divisions, genetic insults typically repaired through HR are instead shunted to backup repair pathways such as alternative end-joining and non-homologous end-joining, leaving behind characteristic genomic DNA repair scars. In 2012, three similar signatures were reported: loss of heterozygosity (LOH), large scale state transitions (LST), and telomeric imbalance (tAI), each characterized by large MBp intra- and interchromosomal rearrangements. These three tests were combined into one genomic readout known commercially as Myriad myChoice CDx HRD score, which is now FDA approved as a companion diagnostic test to select ovarian cancers patients eligible for two PARP inhibitors, Olaparib and Niraparib. While the Myriad myChoice HRD test is currently used to predict PARP inhibitor sensitivity, there is a large overlap between platinum and PARP inhibitor sensitivity as both depend upon inactive HR pathways. Another FDA-approved diagnostic test, FoundationOne CDx, uses LOH and BRCA-status to determine patients eligible for treatment with Olaparib or Rucaparib.

Other HRD signatures subsequently discovered include a base substitution pattern (SBS3) characterized by an even distribution of substitutions without a contextual bias, small deletions with microhomology around flanking the breaksite, and small and large tandem duplications and deletions (RefSig R3/R5). A composite score of SBS3, HRD, indels with microhomology, RefSig R3, and RefSig R5, known as HRDetect (Davies et al., Nat Med 23(4):517-525 (2017); US20200126635), is highly predictive of cases with BRCA1/2 mutations in breast and ovarian cancer genomes. In addition, the small deletion signature was further refined as composed of the ID6 signature, consisting of small deletions of ≥5 bp with small stretches of microhomology in their flanking sequences, and the ID8 signature, primarily associated with germline BRCA1 mutated cases, exhibits deletions of similar size but without microhomology.

The ID6 signature is consistent with repair via alternative end-joining and its predominant mediator polymerase theta (Pol q, gene name POLQ). Repair through Pol θ is also termed Theta Mediated End-joining (TMEJ), a pathway highly used in the absence of functional NHEJ or HR. Loss of POLQ is synthetically lethal with BRCA1 and BRCA2 loss and Pol θ inhibitors are preferentially active in BRCA1 and BRCA2 deleted cell lines. The enzyme typically searches for microhomology within 15 base pairs on either side of the break and utilizes mainly 3 or more base pairs of microhomology. Other pathways, including canonical non-homologous end-joining, can utilize up to 2 bp of microhomology (MH). Thus, a challenge in evaluating TMEJ scars is the degree of overlap between features suggestive of NHEJ and those suggestive of TMEJ. Both NHEJ and TMEJ can utilize short stretches of microhomology up to 2 bp, leaving behind identical deletions.

### NGS Platforms

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLiD sequencing, Ion semiconductor sequencing, Helioscope single molecule sequencing etc.

The Ion Torren™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454TM GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added polyA tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLiD™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

### PARP Inhibitors

In one aspect, the present disclosure provides inhibitory nucleic acids (e.g., sgRNAs, antisense RNAs, ribozymes, or shRNAs) that inhibit PARP expression and/or activity. The mammalian nucleic acid sequences of PARP are known in the art (e.g., NCBI Gene ID: 142). The inhibitory nucleic acids of the present technology may comprise a nucleic acid molecule that is complementary to a portion of a PARP nucleic acid sequence. In some embodiments, the inhibitory nucleic acids (e.g., sgRNAs, antisense RNAs, ribozymes, or shRNAs) target at least one exon and/or intron of PARP. An exemplary nucleic acid sequence of Homo sapiens PARP1 is provided below:

| (SEQ ID NO: 7) | |
|---|---|
| 1 | agcaatctat cagggaacgg cggtggccgg tgcggcgtgt tcggtggcgg ctctggccgc |
| 61 | tcaggcgcct gcggctgggt gagcgcacgc gaggcggcga ggcggcagcg tgtttctagg |
| 121 | tcgtggcgtc gggcttccgg agctttggcg gcagctaggg gaggatggcg gagtcttcgg |
| 181 | ataagctcta tcgagtcgag tacgccaaga gcgggcgcgc ctcttgcaag aaatgcagcg |
| 241 | agagcatccc caaggactcg ctccggatgg ccatcatggt gcagtcgccc atgtttgatg |
| 301 | gaaaagtccc acactggtac cacttctcct gcttctggaa ggtgggccac tccatccggc |
| 361 | accctgacgt tgaggtggat gggttctctg agcttcggtg ggatgaccag cagaaagtca |
| 421 | agaagacagc ggaagctgga ggagtgacag gcaaaggcca ggatggaatt ggtagcaagg |
| 481 | cagagaagac tctgggtgac tttgcagcag agtatgccaa gtccaacaga agtacgtgca |
| 541 | aggggtgtat ggagaagata gaaaagggcc aggtgcgcct gtccaagaag atggtggacc |
| 601 | cggagaagcc acagctaggc atgattgacc gctggtacca tccaggctgc tttgtcaaga |
| 661 | acagggagga gctaggtttc cggcccgagt acagtgcgag tcagctcaag ggcttcagcc |
| 721 | tccttgctac agaggataaa gaagccctga agaagcagct cccaggagtc aagagtgaag |
| 781 | gaaagagaaa aggcgatgag gtggatggag tggatgaagt ggcgaagaag aaatctaaaa |
| 841 | aagaaaaaga caaggatagt aagcttgaaa aagccctaaa ggctcagaac gacctgatct |
| 901 | ggaacatcaa ggacgagcta aagaaagtgt gttcaactaa tgacctgaag gagctactca |
| 961 | tcttcaacaa gcagcaagtg ccttctgggg agtcggcgat cttggaccga gtagctgatg |
| 1021 | gcatggtgtt cggtgccctc cttccctgcg aggaatgctc gggtcagctg gtcttcaaga |
| 1081 | gcgatgccta ttactgcact ggggacgtca ctgcctggac caagtgtatg gtcaagacac |
| 1141 | agacacccaa ccggaaggag tgggtaaccc caaaggaatt ccgagaaatc tcttacctca |
| 1201 | agaaattgaa ggttaaaaaa caggaccgta tattcccccc agaaaccagc gcctccgtgg |
| 1261 | cggccacgcc tccgccctcc acagcctcgg ctcctgctgc tgtgaactcc tctgcttcag |
| 1321 | cagataagcc attatccaac atgaagatcc tgactctcgg gaagctgtcc cggaacaagg |
| 1381 | atgaagtgaa ggccatgatt gagaaactcg gggggaagtt gacggggacg gccaacaagg |
| 1441 | cttccctgtg catcagcacc aaaaaggagg tggaaaagat gaataagaag atggaggaag |
| 1501 | taaaggaagc caacatccga gttgtgtctg aggacttcct ccaggacgtc tccgcctcca |
| 1561 | ccaagagcct tcaggagttg ttcttagcgc acatcttgtc cccttggggg gcagaggtga |
| 1621 | aggcagagcc tgttgaagtt gtggccccaa gagggaagtc aggggctgcg ctctccaaaa |
| 1681 | aaagcaaggg ccaggtcaag gaggaaggta tcaacaaatc tgaaaagaga atgaaattaa |
| 1741 | ctcttaaagg aggagcagct gtggatcctg attctggact ggaacactct gcgcatgtcc |
| 1801 | tggagaaagg tgggaaggtc ttcagtgcca cccttggcct ggtggacatc gttaaaggaa |
| 1861 | ccaactccta ctacaagctg cagcttctgg aggacgacaa ggaaaacagg tattggatat |
| 1921 | tcaggtcctg gggccgtgtg ggtacggtga tcggtagcaa caaactggaa cagatgccgt |
| 1981 | ccaaggagga tgccattgag cacttcatga aattatatga agaaaaaacc gggaacgctt |
| 2041 | ggcactccaa aaatttcacg aagtatccca aaaagttcta ccccctggag attgactatg |
| 2101 | gccaggatga agaggcagtg aagaagctga cagtaaatcc tggcaccaag tccaagctcc |
| 2161 | ccaagccagt tcaggacctc atcaagatga tctttgatgt ggaaagtatg aagaaagcca |
| 2221 | tggtggagta tgagatcgac cttcagaaga tgcccttggg gaagctgagc aaaaggcaga |
| 2281 | tccaggccgc atactccatc ctcagtgagg tccagcaggc ggtgtctcag ggcagcagcg |
| 2341 | actctcagat cctggatctc tcaaatcgct tttacaccct gatcccccac gactttggga |
| 2401 | tgaagaagcc tccgctcctg aacaatgcag acagtgtgca ggccaaggtg gaaatgcttg |
| 2461 | acaacctgct ggacatcgag gtggcctaca gtctgctcag gggagggtct gatgatagca |
| 2521 | gcaaggatcc catcgatgtc aactatgaga agctcaaaac tgacattaag gtggttgaca |
| 2581 | gagattctga agaagccgag atcatcagga agtatgttaa gaacactcat gcaaccacac |
| 2641 | acaatgcgta tgacttggaa gtcatcgata tctttaagat agagcgtgaa ggcgaatgcc |
| 2701 | agcgttacaa gccctttaag cagcttcata accgaagatt gctgtggcac gggtccagga |
| 2761 | ccaccaactt tgctgggatc ctgtcccagg gtcttcggat agccccgcct gaagcgcccg |
| 2821 | tgacaggcta catgtttggt aaagggatct atttcgctga catggtctcc aagagtgcca |
| 2881 | actactgcca tacgtctcag ggagacccaa taggcttaat cctgttggga gaagttgccc |
| 2941 | ttggaaacat gtatgaactg aagcacgctt cacatatcag caagttaccc aagggcaagc |
| 3001 | acagtgtcaa aggtttgggc aaaactaccc ctgatccttc agctaacatt agtctggatg |
| 3061 | gtgtagacgt tcctcttggg accgggattt catctggtgt gaatgacacc tctctactat |
| 3121 | ataacgagta cattgtctat gatattgctc aggtaaatct gaagtatctg ctgaaactga |
| 3181 | aattcaattt taagacctcc ctgtggtaat tgggagaggt agccgagtca cacccggtgg |
| 3241 | ctctggtatg aattcacccg aagcgcttct gcaccaactc acctggccgc taagttgctg |
| 3301 | atgggtagta cctgtactaa accacctcag aaaggatttt acagaaacgt gttaaaggtt |
| 3361 | ttctctaact tctcaagtcc cttgttttgt gttgtgtctg tggggagggg ttgttttggg |
| 3421 | gttgtttttg ttttttcttg ccaggtagat aaaactgaca tagagaaaag gctggagaga |
| 3481 | gattctgttg catagactag tcctatggaa aaaaccaagc ttcgttagaa tgtctgcctt |
| 3541 | actggtttcc ccagggaagg aaaaatacac ttccaccctt ttttctaagt gttcgtcttt |
| 3601 | agttttgatt ttggaaagat gttaagcatt tatttttagt taaaaataaa aactaatttc |
| 3661 | atactattta gattttcttt tttatcttgc acttattgtc ccctttttag ttttttttgt |
| 3721 | ttgcctcttg tggtgagggg tgtgggaaga ccaaaggaag gaacgctaac aatttctcat |
| 3781 | acttagaaac aaaaagagct ttccttctcc aggaatactg aacatgggag ctcttgaaat |
| 3841 | atgtagtatt aaaagttgca tttgaaattc ttgactttct tatgggcact tttgtcttcc |
| 3901 | aaattaaaac tctaccacaa atatacttac ccaagggcta atagtaatac tcgattaaaa |
| 3961 | atgcagatgc cttctcta |

The present disclosure also provides an antisense nucleic acid comprising a nucleic acid sequence that is complementary to and specifically hybridizes with a portion of a PARP mRNA. The antisense nucleic acid may be antisense RNA, or antisense DNA. Antisense nucleic acids based on the known nucleic acid sequences of PARP can be readily designed and engineered using methods known in the art.

Antisense nucleic acids are molecules which are complementary to a sense nucleic acid strand, e.g., complementary to the coding strand of a double-stranded DNA molecule (or cDNA) or complementary to an mRNA sequence. Accordingly, an antisense nucleic acid can form hydrogen bonds with a sense nucleic acid. The antisense nucleic acid can be complementary to an entire PARP coding strand, or to a portion thereof, e.g., all or part of the protein coding region (or open reading frame). In some embodiments, the antisense nucleic acid is an oligonucleotide which is complementary to only a portion of the coding region of PARP mRNA. In certain embodiments, an antisense nucleic acid molecule can be complementary to a noncoding region of the PARP coding strand. In some embodiments, the noncoding region refers to the 5′ and 3′ untranslated regions that flank the coding region and are not translated into amino acids. For example, the antisense oligonucleotide can be complementary to the region surrounding the translation start site of PARP. An antisense oligonucleotide can be, for example, about 5, 10, 15, 20, 25, 30, 35, 40, 45 or 50 nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or modified nucleotides designed to increase the biological stability of the molecules or to increase the physical stability of the duplex formed between the antisense and sense nucleic acids, e.g., phosphorothioate derivatives and acridine substituted nucleotides. Examples of modified nucleotides which can be used to generate the antisense nucleic acid include 5-fluorouracil, 5-bromouracil, 5-chlorouracil, 5-hodouracil, hypoxanthine, xanthine, 4-acetylcytosine, 5-(carboxyhydroxylmethyl)uracil, 5-carboxymethylaminomethyl-2-thouridine, 5-carboxymethylaminometh-yluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-metnylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutosine, pseudouracil, queosine, 2-thiocytosine, 5-methyl-2-thiouracil, 2-thlouracil, 4-thiouracil, 5-methyluracil, uracil-5-oxyacetic acid methylester, uracil-5-cxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules may be administered to a subject or generated in situ such that they hybridize with or bind to cellular mRNA and/or genomic DNA encoding the protein of interest to thereby inhibit expression of the protein, e.g., by inhibiting transcription and/or translation. The hybridization can occur via Watson-Crick base pairing to form a stable duplex, or in the case of an antisense nucleic acid molecule which binds to DNA duplexes, through specific interactions in the major groove of the double helix.

In some embodiments, the antisense nucleic acid molecules are modified such that they specifically bind to receptors or antigens expressed on a selected cell surface, e.g., by linking the antisense nucleic acid molecules to peptides or antibodies which bind to cell surface receptors or antigens. In some embodiments, the antisense nucleic acid molecule is an alpha-anomeric nucleic acid molecule. An alpha-anomeric nucleic acid molecule forms specific double-stranded hybrids with complementary RNA in which, contrary to the usual 0-units, the strands run parallel to each other (Gaultier et al., Nucleic Acids. Res. 15:6625-6641(1987)). The antisense nucleic acid molecule can also comprise a 2′-O -methylribonucleotide (Inoue et al., Nucleic Acids Res. 15:6131-6148 (1987)) or a chimeric RNA-DNA analogue (Inoue et al., FEBS Lett. 215:327-330 (1987)).

The present disclosure also provides a short hairpin RNA (shRNA) or small interfering RNA (siRNA) comprising a nucleic acid sequence that is complementary to and specifically hybridizes with a portion of a PARP mRNA, thereby reducing or inhibiting PARP expression. In some embodiments, the shRNA or siRNA is about 18, 19, 20, 21, 22, 23, 24, 25, 26, 27, 28 or 29 base pairs in length. Double-stranded RNA (dsRNA) can induce sequence-specific post-transcriptional gene silencing (e.g., RNA interference (RNAi)) in many organisms such as C. elegans, Drosophila, plants, mammals, oocytes and early embryos. RNAi is a process that interferes with or significantly reduces the number of protein copies made by an mRNA. For example, a double-stranded siRNA or shRNA molecule is engineered to complement and hybridize to an mRNA of a target gene. Following intracellular delivery, the siRNA or shRNA molecule associates with an RNA-induced silencing complex (RISC), which then binds and degrades a complementary target mRNA (such as PARP mRNA).

The present disclosure also provides a ribozyme comprising a nucleic acid sequence that is complementary to and specifically hybridizes with a portion of a PARP mRNA, thereby reducing or inhibiting PARP expression. Ribozymes are catalytic RNA molecules with ribonuclease activity which are capable of cleaving a complementary single-stranded nucleic acid, such as an mRNA. Thus, ribozymes (e.g., hammerhead ribozymes (described in Haselhoff and Gerlach, Nature 334:585-591 (1988))) can be used to catalytically cleave PARP transcripts, thereby inhibiting translation of PARP.

A ribozyme having specificity for a PARP-encoding nucleic acid can be designed based upon a PARP nucleic acid sequence. For example, a derivative of a Tetrahymena L-19 IVS RNA can be constructed in which the nucleotide sequence of the active site is complementary to the nucleotide sequence to be cleaved in a PARP-encoding mRNA. See, e.g., U.S. Pat. Nos. 4,987,071 and 5,116,742. Alternatively, PARP mRNA can be used to select a catalytic RNA having a specific ribonuclease activity from a pool of RNA molecules. See, e.g., Bartel and Szostak (1993) Science 261:1411-1418, incorporated herein by reference.

The present disclosure also provides a synthetic guide RNA (sgRNA) comprising a nucleic acid sequence that is complementary to and specifically hybridizes with a portion of a PARP nucleic acid sequence. Guide RNAs for use in CRISPR-Cas systems are typically generated as a single guide RNA comprising a crRNA segment and a tracrRNA segment. The crRNA segment and a tracrRNA segment can also be generated as separate RNA molecules. The crRNA segment comprises the targeting sequence that binds to a portion of a PARP nucleic acid sequence, and a stem portion that hybridizes to a tracrRNA. The tracrRNA segment comprises a nucleotide sequence that is partially or completely complementary to the stem sequence of the crRNA and a nucleotide sequence that binds to the CRISPR enzyme. In some embodiments, the crRNA segment and the tracrRNA segment are provided as a single guide RNA. In some embodiments, the crRNA segment and the tracrRNA segment are provided as separate RNAs. The combination of the CRISPR enzyme with the crRNA and tracrRNA make up a functional CRISPR-Cas system. Exemplary CRISPR-Cas systems for targeting nucleic acids, are described, for example, in WO2015/089465.

In some embodiments, a synthetic guide RNA is a single RNA represented as comprising the following elements: 5′-X1-X2-Y-Z-3′ where X1 and X2 represent the crRNA segment, where X1 is the targeting sequence that binds to a portion of a PARP nucleic acid sequence, X2 is a stem sequence that hybridizes to a tracrRNA, Z represents a tracrRNA segment comprising a nucleotide sequence that is partially or completely complementary to X2, and Y represents a linker sequence. In some embodiments, the linker sequence comprises two or more nucleotides and links the crRNA and tracrRNA segments. In some embodiments, the linker sequence comprises 2, 3, 4, 5, 6, 7, 8, 9, 10 or more nucleotides. In some embodiments, the linker is the loop of the hairpin structure formed when the stem sequence hybridized with the tracrRNA.

In some embodiments, a synthetic guide RNA is provided as two separate RNAs where one RNA represents a crRNA segment: 5′-X1-X2-3′ where X1 is the targeting sequence that binds to a portion of a PARP nucleic acid sequence, X2 is a stem sequence that hybridizes to a tracrRNA, and one RNA represents a tracrRNA segment, Z, that is a separate RNA from the crRNA segment and comprises a nucleotide sequence that is partially or completely complementary to X2 of the crRNA.

Exemplary crRNA stem sequences and tracrRNA sequences are provided, for example, in WO/2015/089465, which is incorporated by reference herein. In general, a stem sequence includes any sequence that has sufficient complementarity with a complementary sequence in the tracrRNA to promote formation of a CRISPR complex at a target sequence, wherein the CRISPR complex comprises the stem sequence hybridized to the tracrRNA. In general, degree of complementarity is with reference to the optimal alignment of the stem and complementary sequence in the tracrRNA, along the length of the shorter of the two sequences. Optimal alignment may be determined by any suitable alignment algorithm, and may further account for secondary structures, such as self-complementarity within either the stem sequence or the complementary sequence in the tracrRNA. In some embodiments, the degree of complementarity between the stem sequence and the complementary sequence in the tracrRNA along the length of the shorter of the two when optimally aligned is about or more than about 25%, 30%, 40%, 50%, 60%, 70%, 80%, 90%, 95%, 97.5%, 99%, or higher. In some embodiments, the stem sequence is about or more than about 5, 6, 7, 8, 9, 10, 11, 12, 13, 14, 15, 16, 17, 18, 19, 20, 25, 30, 40, 50, or more nucleotides in length. In some embodiments, the stem sequence and complementary sequence in the tracrRNA are contained within a single RNA, such that hybridization between the two produces a transcript having a secondary structure, such as a hairpin. In some embodiments, the tracrRNA has additional complementary sequences that form hairpins. In some embodiments, the tracrRNA has at least two or more hairpins. In some embodiments, the tracrRNA has two, three, four or five hairpins. In some embodiments, the tracrRNA has at most five hairpins.

In a hairpin structure, the portion of the sequence 5′ of the final “N” and upstream of the loop corresponds to the crRNA stem sequence, and the portion of the sequence 3′ of the loop corresponds to the tracrRNA sequence. Further non-limiting examples of single polynucleotides comprising a guide sequence, a stem sequence, and a tracr sequence are as follows (listed 5′ to 3′), where “N” represents a base of a guide sequence (e.g. a modified oligonucleotide provided herein), the first block of lower case letters represent stem sequence, and the second block of lower case letters represent the tracrRNA sequence, and the final poly-T sequence represents the transcription terminator:

(a)
(SEQ ID NO: 1)
NNNNNNNNNNNNNNNNNNNNgtttttgtactctcaagatttaGAAAtaa
atcttgcagaagctacaaagataaggcttcatgccgaaatcaacaccct
gtcattttatggcagggtgttttcgttatttaaTTTTTT;
(b)
(SEQ ID NO: 2)
NNNNNNNNNNNNNNNNNNNNgtttttgtactctcaGAAAtgcagaagct
acaaagataaggcttcatgccgaaatcaacaccctgtcattttatggca
gggtgttttcgttatttaaTTTTTT;
(c)
(SEQ ID NO: 3)
NNNNNNNNNNNNNNNNNNNNgtttttgtactctcaGAAAtgcagaagct
acaaagataaggcttcatgccgaaatcaacaccctgtcattttatggca
gggtgtTTTTTT;
(d)
(SEQ ID NO: 4)
NNNNNNNNNNNNNNNNNNNNgttttagagctaGAAAtagcaagttaaaa
taaggctagtccgttatcaacttgaaaaagtggcaccgagtcggtgcTT
TTTT;
(e)
(SEQ ID NO: 5)
NNNNNNNNNNNNNNNNNNNNgttttagagctaGAAATAGcaagttaaaa
taaggctagtccgttatcaacttgaaaaagtgTTTTTTT;
and
(f)
(SEQ ID NO: 6)
NNNNNNNNNNNNNNNNNNNNgttttagagctagAAATAGcaagttaaaa
taaggctagtccgttatcaTTTTTTTT.

Selection of suitable oligonucleotides for use as a targeting sequence in a CRISPR Cas system depends on several factors including the particular CRISPR enzyme to be used and the presence of corresponding proto-spacer adjacent motifs (PAMs) downstream of the target sequence in the target nucleic acid. The PAM sequences direct the cleavage of the target nucleic acid by the CRISPR enzyme. In some embodiments, a suitable PAM is 5′-NRG or 5′-NNGRR (where N is any Nucleotide) for SpCas9 or SaCas9 enzymes (or derived enzymes), respectively. Generally, the PAM sequences should be present between about 1 to about 10 nucleotides of the target sequence to generate efficient cleavage of the target nucleic acid. Thus, when the guide RNA forms a complex with the CRISPR enzyme, the complex locates the target and PAM sequence, unwinds the DNA duplex, and the guide RNA anneals to the complementary sequence on the opposite strand. This enables the Cas9 nuclease to create a double-strand break.

A variety of CRISPR enzymes are available for use in conjunction with the disclosed guide RNAs of the present disclosure. In some embodiments, the CRISPR enzyme is a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas9 protein, for instance any naturally-occurring bacterial Cas9 as well as any chimeras, mutants, homologs or orthologs. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof. In some embodiments, the CRISPR enzyme cleaves both strands of the target nucleic acid at the Protospacer Adjacent Motif (PAM) site. In some embodiments, the CRISPR enzyme is a nickase, which cleaves only one strand of the target nucleic acid.

In another aspect, the present disclosure provides pharmacological inhibitors of PARP including, but not limited to iniparib, olaparib, niraparib, rucaparib, talazoparib, veliparib, AG014699, CEP 9722, MK 4827, BMN-673, E7016, or 3-aminobenzamide. Anti-PARP neutralizing antibodies may also be employed in the methods disclosed herein.

### Pharmaceutical Compositions Including the PARP Inhibitors and Platinum Agents

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Modes of Administration and Effective Dosages

Any method known to those in the art for contacting a cell, organ or tissue with one or more PARP inhibitors and/or platinum agents disclosed herein may be employed. Suitable methods include in vitro, ex vivo, or in vivo methods. In vivo methods typically include the administration of one or more PARP inhibitors and/or platinum agents to a mammal, suitably a human. When used in vivo for therapy, the one or more PARP inhibitors and/or platinum agents described herein are administered to the subject in effective amounts (i.e., amounts that have desired therapeutic effect). The dose and dosage regimen will depend upon the degree of the disease state of the subject, the characteristics of the particular PARP inhibitor or platinum agent used, e.g., its therapeutic index, and the subject's history.

The effective amount may be determined during pre-clinical trials and clinical trials by methods familiar to physicians and clinicians. An effective amount of one or more PARP inhibitors and/or platinum agents useful in the methods may be administered to a mammal in need thereof by any of a number of well-known methods for administering pharmaceutical compounds. The PARP inhibitor or platinum agent may be administered systemically or locally.

The one or more PARP inhibitors and/or platinum agents described herein can be incorporated into pharmaceutical compositions for administration, singly or in combination, to a subject for the treatment or prevention of a disease or condition described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more PARP inhibitors and/or platinum agents disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly a-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more PARP inhibitors and/or platinum agents disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more PARP inhibitor/platinum agent concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more PARP inhibitors and/or platinum agents may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Methods for Treatment Selection in HRD Cancer Patients

In one aspect, the present disclosure provides a method for selecting a cancer patient for treatment with a PARP inhibitor or a platinum agent comprising: (a) detecting levels of templated insertions (TINS) in a biological sample obtained from the cancer patient that are elevated relative to a control sample obtained from a healthy subject or a predetermined threshold; and (b) administering to the cancer patient an effective amount of a PARP inhibitor or a platinum agent. In another aspect, the present disclosure provides a method for prolonging survival of a cancer patient comprising administering to the cancer patient an effective amount of a PARP inhibitor or a platinum agent, wherein levels of templated insertions (TINS) in a biological sample obtained from the cancer patient are elevated relative to a control sample obtained from a healthy subject or a predetermined threshold. In some embodiments of the methods disclosed herein, the levels of TINS are detected via next-generation sequencing.

In any of the preceding embodiments of the methods disclosed herein, the TINS comprise direct-repeat TINS (drTINS) and/or inverted TINS (iTINS). Additionally or alternatively, in some embodiments of the methods disclosed herein, the TINS (e.g., drTINS and/or iTINS) have a length ranging from 5 base pairs (bps) to 20 base pairs (bps) or 20 base pairs (bps) to 50 base pairs (bps). In certain embodiments, the length of the TINS (e.g., drTINS and/or iTINS) is 5 bps, 6 bps, 7 bps, 8 bps, 9 bps, 10 bps, 11 bps, 12 bps, 13 bps, 14 bps, 15 bps, 16 bps, 17 bps, 18 bps, 19 bps, 20 bps, 21 bps, 22 bps, 23 bps, 24 bps, 25 bps, 26 bps, 27 bps, 28 bps, 29 bps, 30 bps, 31 bps, 32 bps, 33 bps, 34 bps, 35 bps, 36 bps, 37 bps, 38 bps, 39 bps, 40 bps, 41 bps, 42 bps, 43 bps, 44 bps, 45 bps, 46 bps, 47 bps, 48 bps, or 49 bps.

Additionally or alternatively, in some embodiments, the cancer patient is diagnosed with, or is at risk of having a homologous recombination deficiency (HRD). In certain embodiments, the HRD comprises mutations that result in the inactivation of at least one of the following genes: PALP2/FANCN, BRIP1/FANCJ, BARD1, RAD51, RAD51 paralogs (RAD51B, RAD51C, RAD51D, XRCC2, XRCC3), FANCA, FANCB, FANCC, FANCD2, FANCE, FANCG, FANCI, FANCL, FANCM, FAN1, SLX4/FANCP and ERCC1. In any and all embodiments of the methods disclosed herein, the cancer patient harbors a mutation in one or more driver mutations selected from among BRCA2, BRCA1, IDH1, CDK4 and RB1. Additionally or alternatively, in some embodiments, the cancer patient is suffering from a cancer selected from among breast cancer, ovarian cancer, prostate cancer, and pancreatic cancer. In certain embodiments, the cancer patient exhibits stage I, stage II, stage III or stage IV cancer. Additionally or alternatively, in some embodiments, the cancer patient has not received at least one prior line of anti-cancer therapy. In other embodiments, the cancer patient has received at least one prior line of anti-cancer therapy. Examples of anti-cancer therapy include chemotherapy, radiation therapy, surgery or any combination thereof.

In any and all embodiments of the methods disclosed herein, the PARP inhibitor is a PARP-specific inhibitory nucleic acid (e.g., sgRNAs, antisense RNAs, ribozymes, or shRNAs), iniparib, olaparib, niraparib, rucaparib, talazoparib, veliparib, AG014699, CEP 9722, MK 4827, BMN-673, E7016, 3-aminobenzamide, or an anti-PARP neutralizing antibody.

In any and all embodiments of the methods disclosed herein, the platinum agent is cisplatin, carboplatin, oxaliplatin, nedaplatin, triplatin tetranitrate, phenanthriplatin, pyriplatin, picoplatin, or satraplatin.

Additionally or alternatively, in some embodiments of the methods disclosed herein, the biological sample obtained from the cancer patient comprises biopsied tumor tissue, whole blood, plasma, or serum.

Once treated for a particular period of time (e.g., between one to six months), the patient can be assessed to determine whether or not the treatment regimen has an effect. If a beneficial effect is detected, the patient can continue with the same or a similar cancer treatment regimen. If a minimal or no beneficial effect is detected, then adjustments to the cancer treatment regimen can be made. For example, the dose, frequency of administration, or duration of treatment can be increased. In some cases, additional anti-cancer agents can be added to the treatment regimen or a particular anti-cancer agent can be replaced with one or more different anti-cancer agents. The patient being treated can continue to be monitored as appropriate, and changes can be made to the cancer treatment regimen as appropriate.

Additionally or alternatively, in some embodiments, the methods of the present technology further comprise determining an additional HRD signature in the cancer patient. In some embodiments, the additional HRD signature comprises one or more of loss of heterozygosity (LOH), large scale state transitions (LST), and telomeric imbalance (tAI). In certain embodiments, the additional HRD signature comprises one or more of single base substitution signature 3 (SBS3), single base substitution signature 8 (SBS8), short tandem duplications (RefSig R3), short deletions (RefSig R5), microhomology-mediated indels, and HRD score. In other embodiments, the additional HRD signature comprises ID8 signature, and ID6 signature.

### EXAMPLES

The present technology is further illustrated by the following Examples, which should not be construed as limiting in any way. The examples herein are provided to illustrate advantages of the present technology and to further assist a person of ordinary skill in the art with preparing or using the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Materials and Methods

Driver Calls: Mutational signature analyses were conducted with data from The Pan-Cancer Analysis of Whole Genomes (PCAWG), a consortium of the International Cancer Genome Consortium (ICGC) and The Cancer Genome Atlas (TCGA) (dcc.icgc.org/releases/PCAWG, accessed August 2020). PCAWG had 2,793 whole cancer genomes available for analysis. Of these 2,793 samples, 2,354 had cancer driver mutation calls and were publicly available. These include previously known driver gene single nucleotide variants, indels, structural variants and translocations, non-coding, and non-genic elements totaling 674 unique driver alterations. Only drivers with over ten samples were tested to ensure enough data for continued analysis, leaving 219 drivers available for study.

Known Mutational Signatures: The 2,354 PCAWG genomes with driver calls were aligned with single base substitution (SBS) and indel (ID) signature calls generated by Alexandrov et al., Nature 578, 94-101 (2020), using SigProfiler based on sample id numbers. Proportions of SBS3, ID6, and ID8 signatures were calculated for each sample. To understand the portion of double-strand break repair events reflected in ID6 and ID8, specific indel signatures known to reflect other DNA repair events were accounted for. ID1 and ID2 mutations were excluded as they are caused by slippage events during DNA replication. Likewise, ID7 mutations caused by defective DNA mismatch repair and ID13 mutations resulting from DNA damage induced by UV light were omitted. ID11 and ID16 were found to be predominately insertions and were also omitted.

Large-scale state transitions (LST), loss of heterozygosity (LOH), and telomere allelic imbalances (TAI), all markers of HRD, were determined for all samples using a modified version of the calc.lsto, calc.loho, and calc.aio functions from the Signature Tools Lib R package, respectively. HRD score was determined by computing the unweighted sum of LST, TAI, and LOH. HRDetect probabilities were calculated using the HRDetect pipeline from the Signature Tools Lib R package. For HRDetect calculations, 42 samples were missing BEDPE files, and HRDetect probabilities could not be computed and thus were left out of future analyses.

Novel Mutational Profiles: Indel profiles were generated consisting of insertions and deletions sizes, repeats, and microhomology lengths. VCF files of PCAWG samples were run through a combination of the indel caller within the HRDetect toolbox and previously developed tools to check for sequence context around indels and determine the presence of microhomology or repetitive regions. Novel signatures were defined using these profiles and to create TMEJ specific signatures. These novel TMEJ-specific signatures have 1-30 bp deletions with ≥2 (TMEJ2), ≥3 (TMEJ3), or ≥4 (TMEJ4) bp of microhomology. TMEJ deletions by the signatures were normalized by the total number of indels to standardize the proportion of these events.

Templated insertions (TINS) were identified according to a protocol developed by Carvajal-Garcia et al., Proc Natl Acad Sci USA 117, 8476-8485 (2020), which scans for direct or inverted repeats within 50 bp on either side of insertions of length 5 or larger. To ensure no tandem repeats were included, insertions directly adjacent to their template, or a 0 bp distance between the insertion and templated sequence, were removed from further analysis. Human genome build hs37d5 was used as a reference genome (as used in PCAWG variant analyses). Inverted and direct repeats were summed to create a total TINS count. The unique categories of inverted TINS (iTINS) and direct repeat TINS (drTINS) were tallied separately. Raw TINS, iTINS, and drTINS counts were normalized by the total indel count to create comparable frequency counts across samples.

Statistical Analysis: Univariate Mann-Whitney U-tests were used to test each of 219 cancer driver mutations for enrichment in each of the previously defined mutational signatures by comparing mutated samples to wild-type for every driver mutation. Significant driver hits (false discovery rate≤0.05) were passed into the multivariate analyses. Multivariate analyses were performed using linear regression on each mutational signature with univariate significant driver hits and cancer type as covariates. All analyses were performed in R (version 4.0.3). Statistical tests were considered significant as P≤0.05 (or FDR≤0.05). Asterisks used to define significance as follows: *P≤0.05, **P≤0.01, ***, P≤0.001, ****P≤0.0001.

Survival Analysis: Survival time is defined as the time interval between diagnosis to death or last follow-up. All survival analyses were performed on either PCAWG stage III and IV ovarian cancer cases (106 samples) or TCGA stage III and IV, platinum-treated ovarian cancer cases (407 samples). Kaplan-Meier curves were generated and compared using log-rank tests for PCAWG stage III and IV ovarian cancer cases for HRD and HR competent groups depending on mutational signatures. Univariate Cox proportional hazards regression models were fitted with each cancer driver as the predictor for PCAWG stage III and IV ovarian cancer cases and TCGA stage III and IV, platinum-treated ovarian cancer cases. In addition, the relationship between mutational signatures and survival was examined by fitting a Cox proportional hazards model.

Mutational signatures were dichotomized to indicate HRD cases based on each signature. When available, known signature thresholds were used to create distinct cutoffs. HRDetect probability of ≥0.7 and HRD score≥42 have been previously reported as acceptable HRD thresholds. There are no known thresholds for SBS3, ID6, or ID8, so any signature presence was considered an HRD threshold. The median value of the signature for PCAWG stage III and IV ovarian cancer cases were used to determine the HRD cutoff for the novel signatures. For TMEJ these values are 0.03 for TMEJ2, 0.01 for TMEJ3, and 0.003 for TMEJ4. For TINS, 0.007 for TINS and 0.003 for iTINS and drTINS.

### Example 2: Known Signatures do not Demonstrate any Novel Associations with PCAWG Driver Mutations

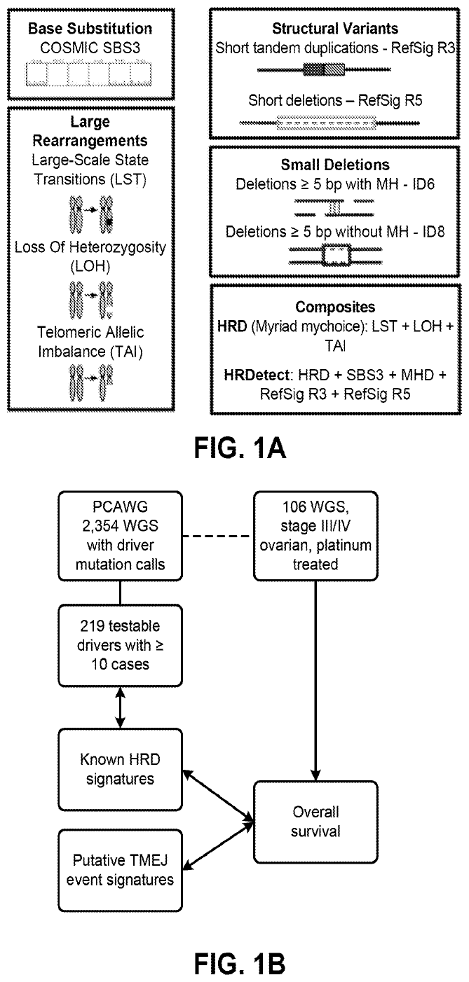

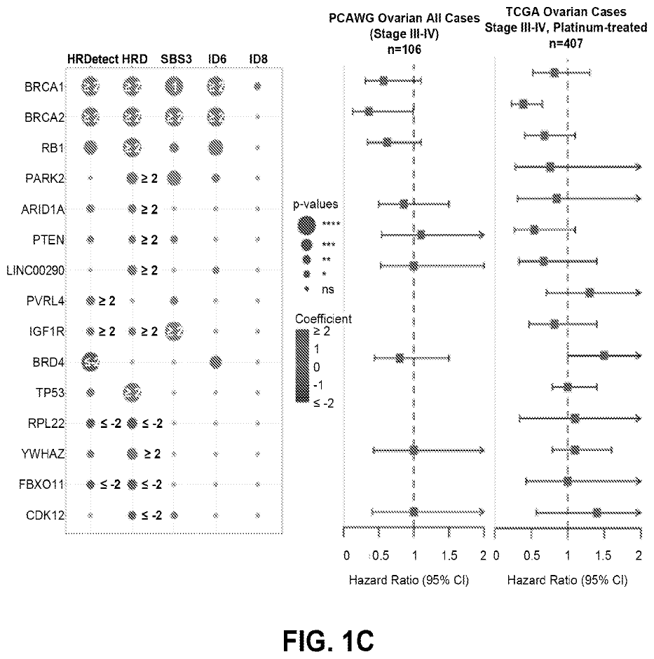

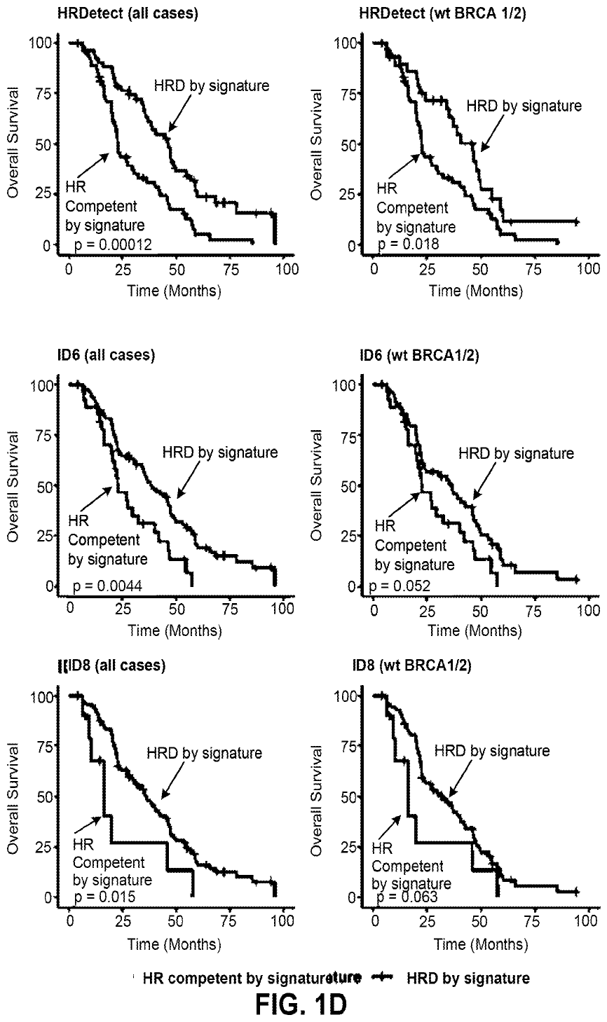

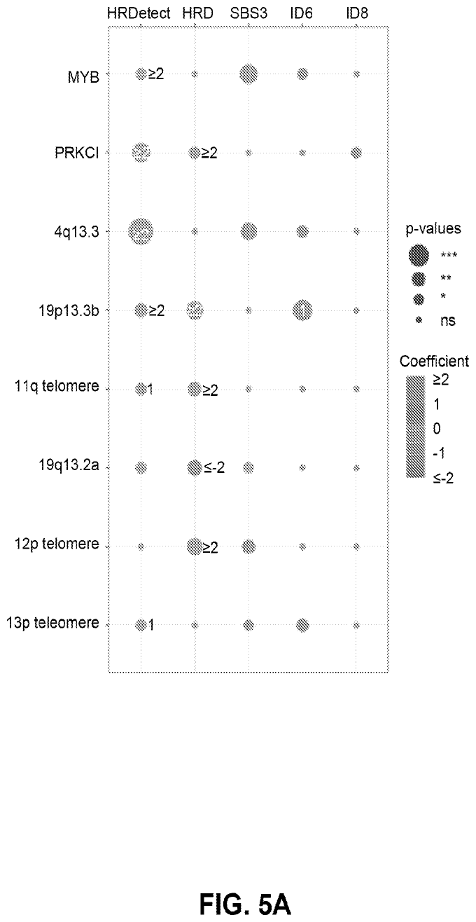

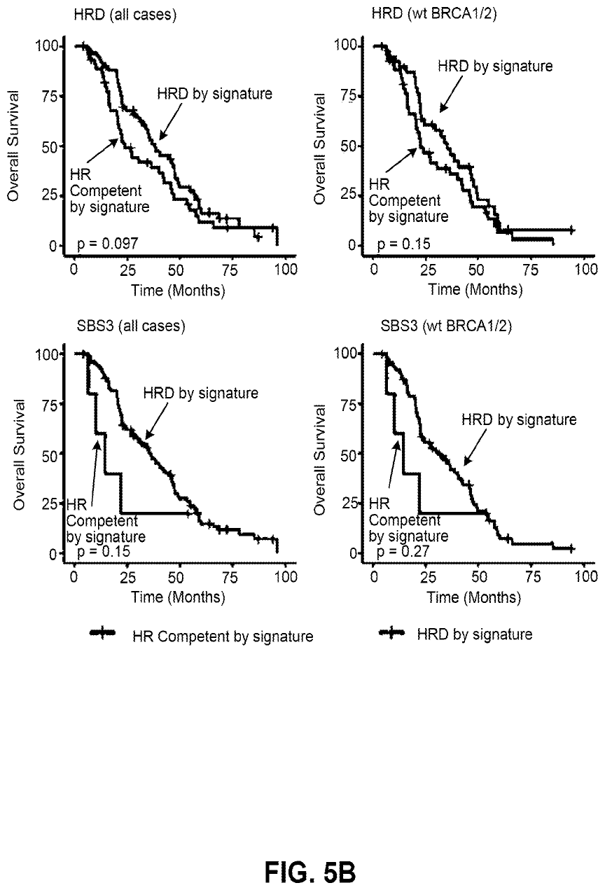

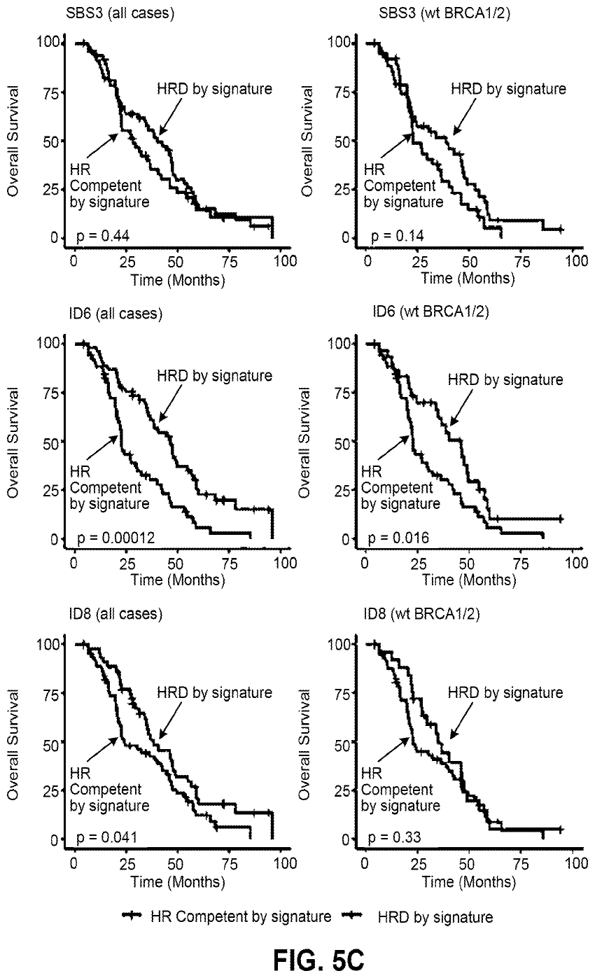

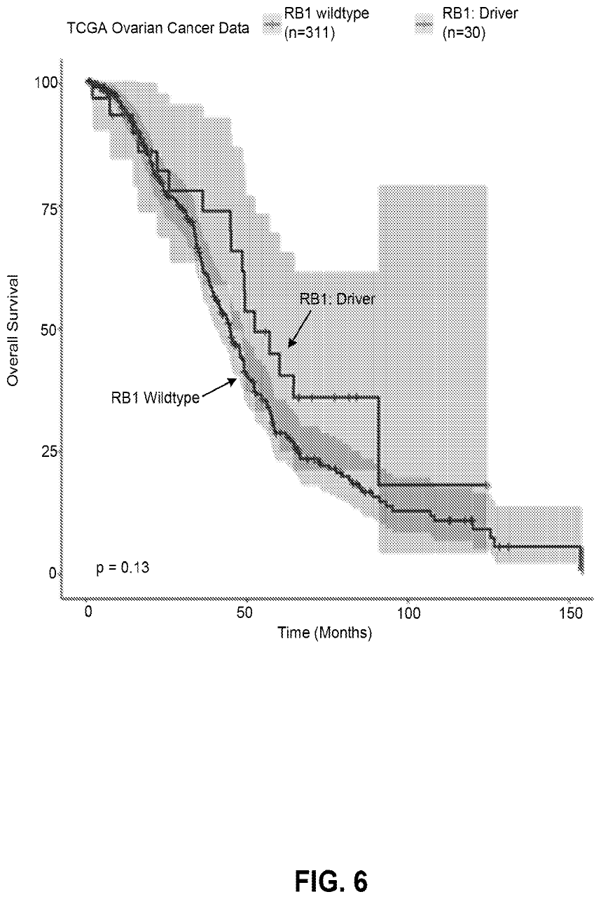

Homologous recombination deficiency (HRD) in the face of genomic insults creates various genomic scars reflective of the DNA repair pathway used. Using whole genomes from The Pan-Cancer Analysis of Whole Genomes (PCAWG) project, known HRD signatures, including base substitutions, large rearrangements, structural variants, small indels, and composite HRD scores were analyzed (FIG. 1A). The relationship between these signatures and driver mutations previously called for each case in PCAWG, including single-gene mutations, copy number alterations, long non-coding RNAs, and other non-coding driver events were examined (FIGS. 1B, 1C, 5A, 5B, 5C). The dataset contains 219 testable drivers (present in at least 10 genomes) called in 2,354 cases.

Using univariate Mann-Whitney U-tests, the relationship between the presence of a driver and the HRD signature were determined (FIG. 1B). Significant drivers in univariate analysis (FDR<0.05) for each signature were then applied to a multivariate linear regression model to account for the co-occurrence of driver genes. Cancer type was also included in the multivariate analysis as per previous analyses to account for inherently different baselines of genomic alteration. Pathogenic biallelic BRCA1 and BRCA2 driver mutations had the most significant associations with all HRD signatures (FIG. 1C, FIG. 5A). RB1 loss was also associated with four of the five tested signatures, albeit more weakly. The remaining tested drivers were inconsistently associated with each signature, suggesting non-specificity.

The HRD detection performance of driver mutations were evaluated using patient survival data from stage III/IV platinum-treated ovarian cancers as a clinical surrogate of HR deficiency. Survival data matched to whole genomes in the PCAWG (n=106) and exomes/whole genomes in the TCGA (n=407) are shown. Of those testable drivers (significant in at least two multivariate analyses), only pathogenic driver mutations in BRCA1 and BRCA2 had consistent, significant associations with the signatures and patient survival (FIG. 1C). While RB1 appeared to have a strong association with four of the five signatures, there was no significant difference in survival between patients with and without RB1 driver mutations (FIG. 1C, FIG. 5A). Similarly, there was not a significant association with survival among patients with BRD4 amplification (FIG. 1C) though there is a strong negative association with HRDetect. Therefore, it is not likely that other clearly identifiable drivers in non-HR genes would provide prognostic value for HR capacity and platinum sensitivity in this dataset. Other altered core HR genes beyond BRCA1/2 mutations, including PALB2 (5 cases) and RAD51C (3 cases), were rare.

The prognostic value of the signatures themselves were then determined, both in all cases and only in the BRCA1/2 wild-type cases, using survival analyses per Kaplan-Meier survival curves and log-rank tests. For HRD score and HRDetect analyses, patients were grouped into HR competent or deficient by using established thresholds. Significant differences in survival were observed between the two groups defined by HRDetect but not HRD score (FIG. 1D and FIG. 5B). The SBS3, ID6, and ID8 signatures do not have established thresholds for determining HR competency vs. deficiency. Thus, signature presence or absence was used as a binary factor. In addition, a threshold set at the median SBS3, ID6, or ID8 contribution was assessed across all cases. ID6 and ID8 separated high and low-risk cohorts, including all cases, but only ID6 remained marginally significant in BRCA1/2 wild-type cases (FIG. 1D and FIG. 5C).

### Example 3: Novel TMEJ Deletion Signature Performs Similarly to ID6

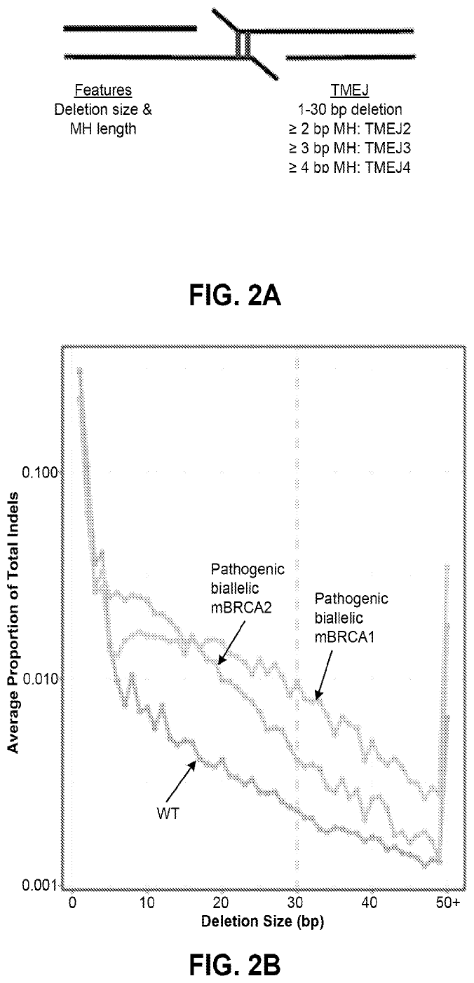

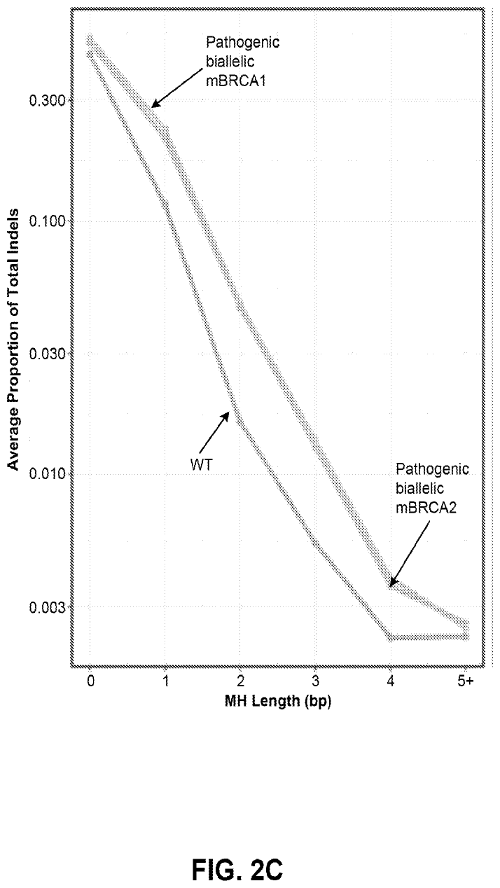

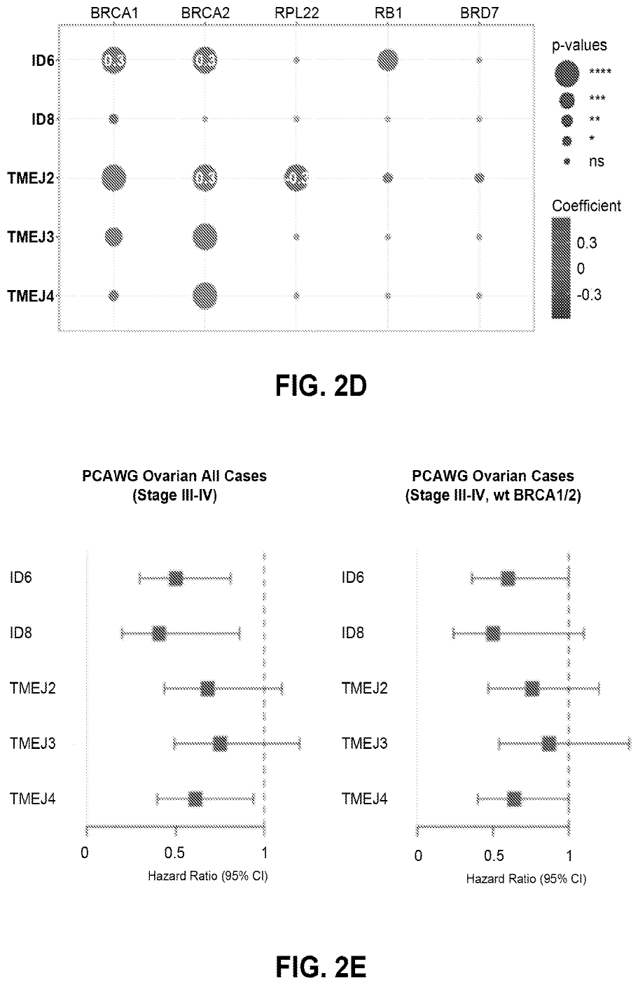

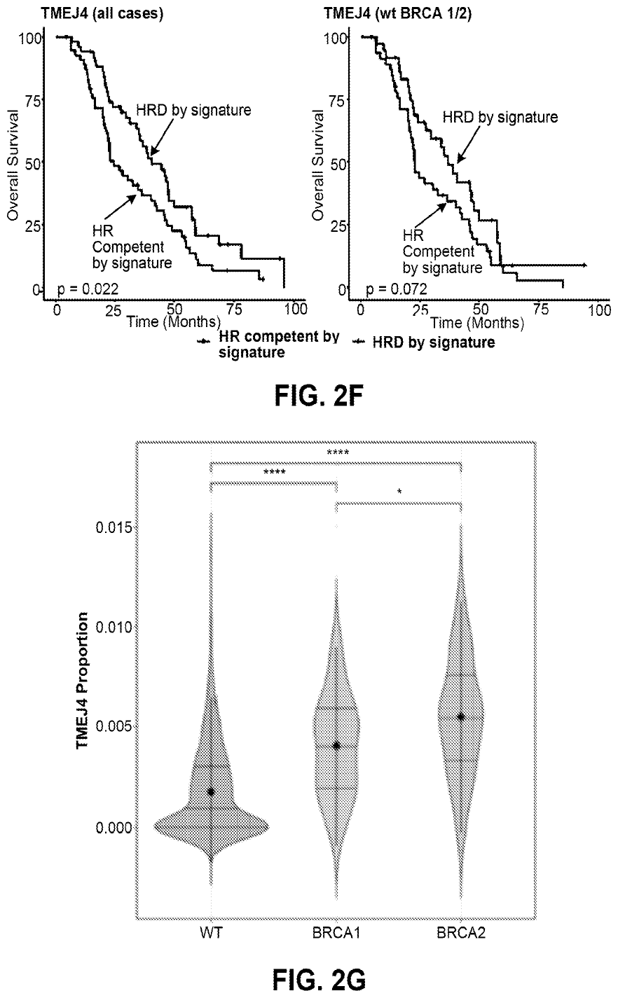

Given the limited utility of existing driver mutations and associated signatures to predict HRD, attempts were made to develop a novel signature based on known markers caused by theta-mediated end-joining (TMEJ). TMEJ is classified by the use of polymerase theta (Pol θ) to repair stranded double-strand breaks using small stretches of microhomology to align and repair breaks, often resulting in small deletions (FIG. 2A). After a double-strand break and resection of the 5′ ends, Pol θ utilizes microhomology (MH) preferentially within 15 bp on either side of the break to align and anneal the two strands. An analysis of deletion sizes showed that deletions in the 5-30 bp range were increased in pathogenic, biallelically mutated BRCA1, BRCA2 samples compared to wild-type PCAWG samples (FIG. 2B). Similarly, indels with 1-4 bp of microhomology were much more common among BRCA1 and BRCA2 mutated samples than wild-type (FIG. 2C). This is in line with prior studies, which reported that Pol θ preferentially uses 2-6 bp of microhomology. The ID6 signature represents small deletions >5kb and predominantly>2 bp of MH. In recognition of the typical size of TMEJ scars seen preclinically and the possibility of overlap of NHEJ and TMEJ scars with short MH stretches, three TMEJ signatures were tested, defined as deletions of 1-30 bp with microhomology lengths starting from 2 to 4 bp (TMEJ2-TMEJ4).

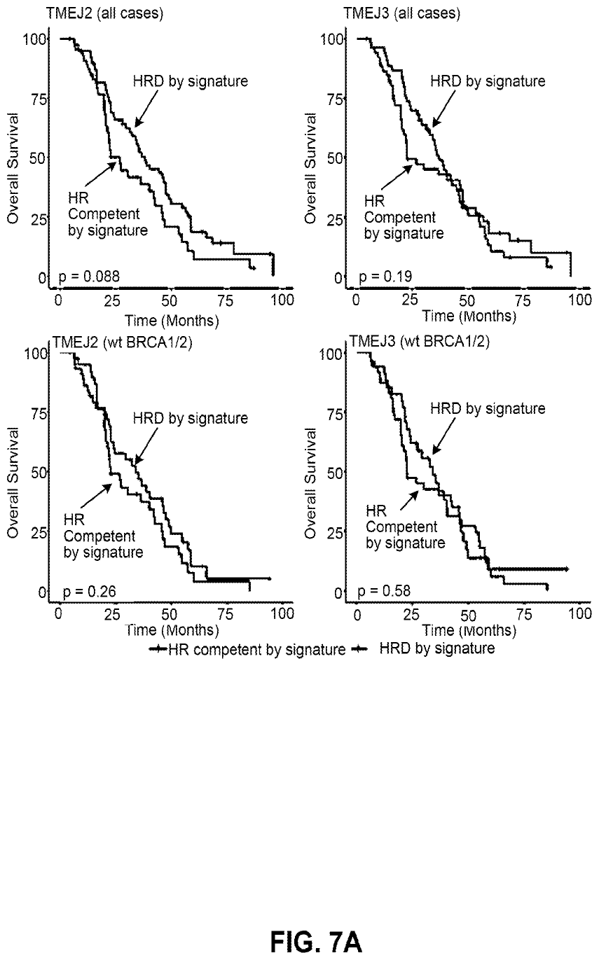

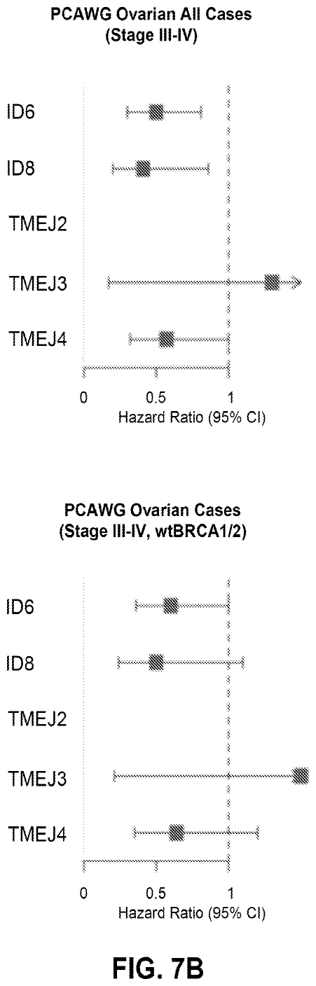

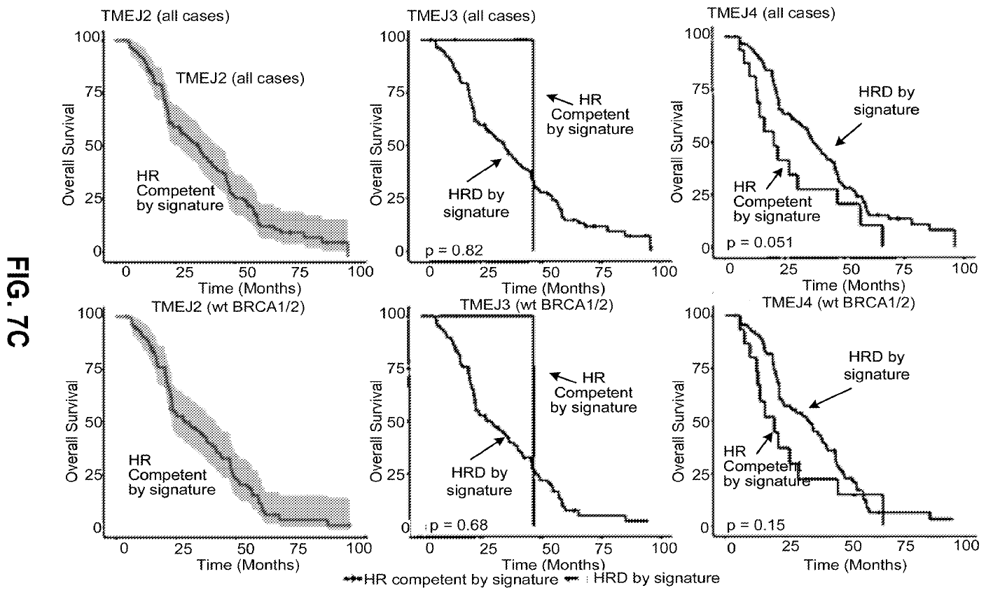

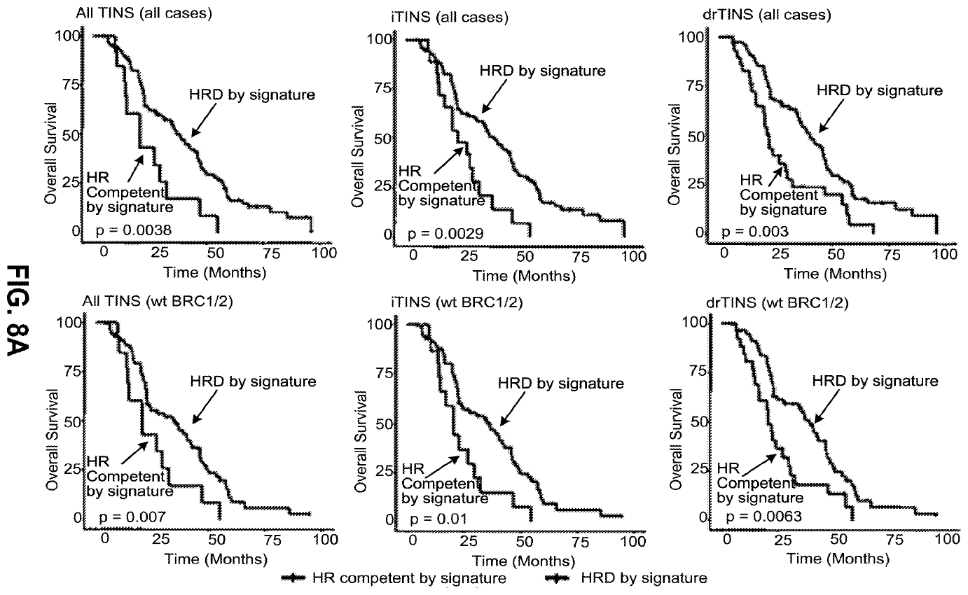

The same univariate and multivariate analysis approach was performed with the 219 unique driver mutations and the novel TMEJ signatures. BRCA1 and BRCA2 mutations had the most significant associations with the novel signatures, BRCA1 becoming gradually less significant as the microhomology size increased while BRCA2 remained consistently significantly associated (FIG. 2D). Using the median values of each signature as a threshold, univariate cox regression analysis was performed and Kaplan-Meier survival curves were generated with log-rank test comparisons in the PCAWG stage III and IV platinum-treated ovarian cancer cohort. It was found that while TMEJ2 and TMEJ3 are not associated with improved survival, TMEJ4 performs similarly to ID6 in separating high and low-risk groups but does not reach statistical significance (FIGS. 2E, 2F, 7A). In this analysis, 0 was also considered as a threshold to generate binary groups: any count of these events versus no counts, as performed in FIG. 1 for SBS3, ID6, and ID8. However, it was noted that due to the high frequency of TMEJ2-3 events, few genomes were without at least one such deletion (FIGS. 7B, 7C). In addition to being associated with improved survival, TMEJ4 was increased in pathogenic, biallelic mutated BRCA1 and BRCA2 compared to wild-type in breast, prostate, ovarian, and pancreas cancer samples (FIG. 2G). Thus, we conclude that TMEJ4 performs similarly to ID6, though the TMEJ4 criteria are closer to what is known pre-clinically about TMEJ repair.

### Example 4: Templated Insertions (TINS) Associate with BRCA2 Mutations and Overall Survival in Advanced Ovarian Cancers Treated with Platinum Agents

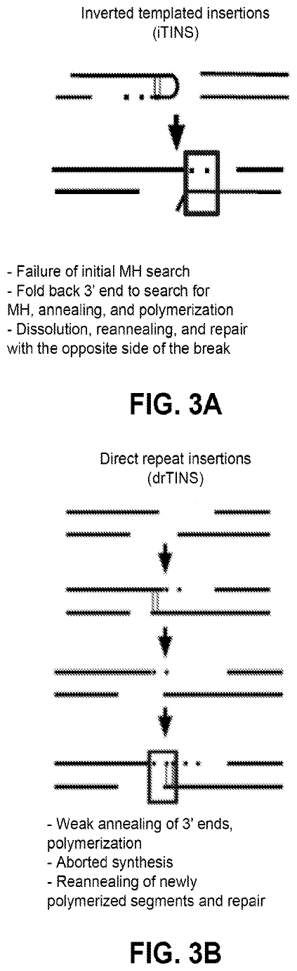

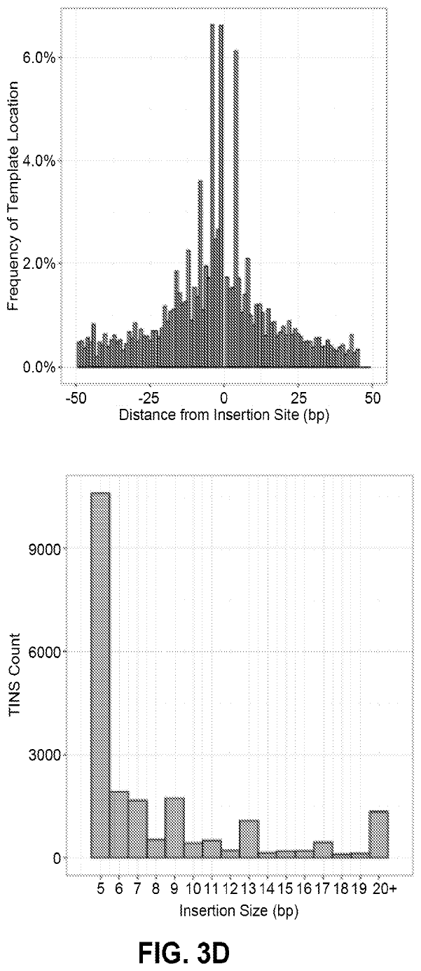

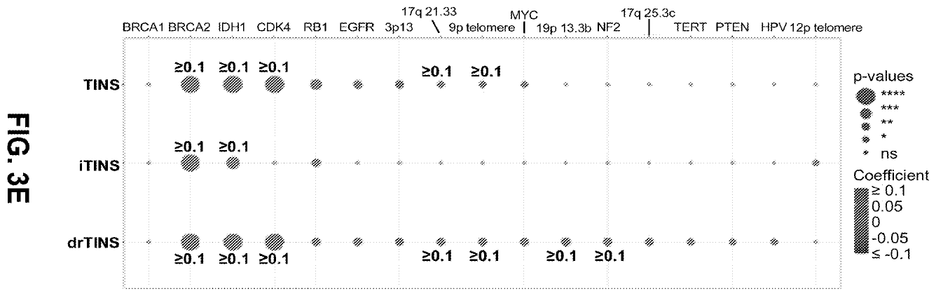

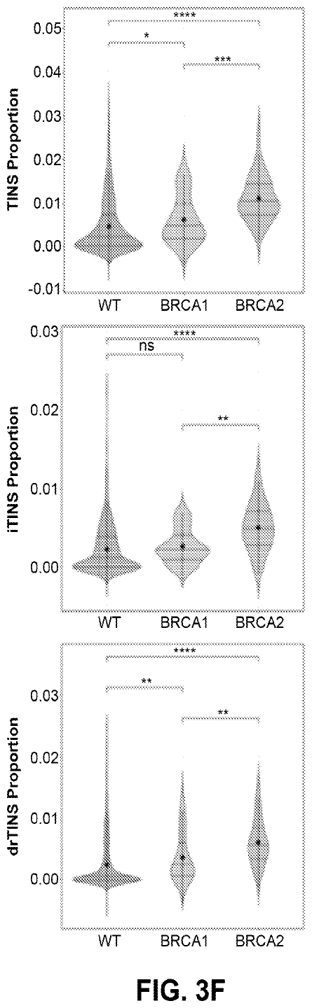

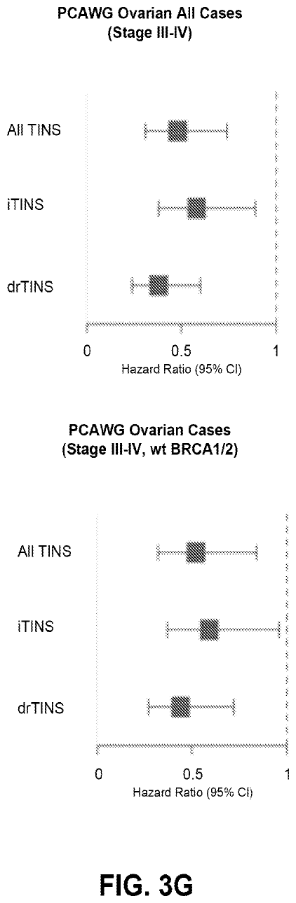

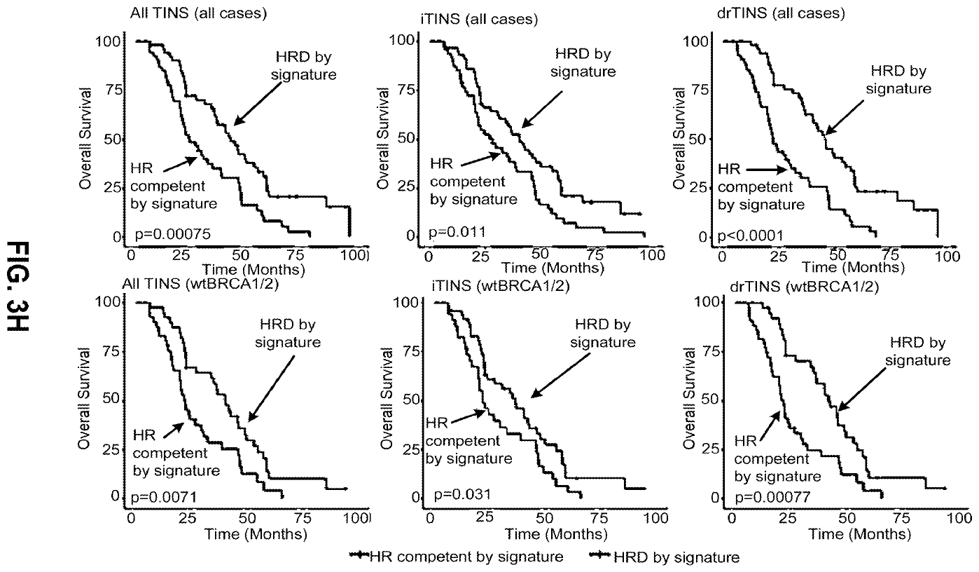

Another unique characteristic of TMEJ is small, templated insertions (TINS). These insertions include two categories of direct-repeat templated insertions (drTINS) and inverted templated insertions (iTINS) (FIGS. 3A, 3B). When there is insufficient microhomology between DNA strands on opposite sides of the break, 3′ overhangs can fold back on themselves and find microhomology to begin fill-in synthesis (FIG. 3A), followed by dissolution, re-annealing across the break, and repair. iTINS are then insertions≥5 bp insertions that are reverse complements of the neighboring sequence, 50 bp on either side of the insertion site. drTINS are insertions of >5 bp that are direct repeats of the adjacent sequence within 50 bp on either side of the insertion site. These events result from the initial microhomology annealing between the two DNA strands slipping and reannealing after fill-in synthesis has begun (FIG. 3B). The 5 bp cutoff was used to decrease the probability of finding these insertion events by chance and removed from analysis insertions at tandem repeats, as shown previously (Carvajal-Garcia, J. et al. Proc Natl Acad Sci USA 117, 8476-8485 (2020). The templates for the insertions were predominantly located directly adjacent to the insertions themselves (FIG. 3C). Most of the TINS insertions are 5-6 bp insertions in length, but larger drTINS insertions are also present (FIG. 3D).

The same univariate and multivariate analysis approach on the three TINS signatures, iTINS, drTINS, and the combination of the two. Both all TINS and drTINS had possibly non-specific associations with various driver mutations (FIG. 3E). All three signatures, TINS, iTINS, and drTINS, were significantly associated with BRCA2 driver mutations in a pan-cancer context and were increased in pathogenic, biallelic mutated BRCA2 compared to BRCA1 and wild type in breast, prostate, ovarian, and pancreas cancers (FIGS. 3E, 3F).

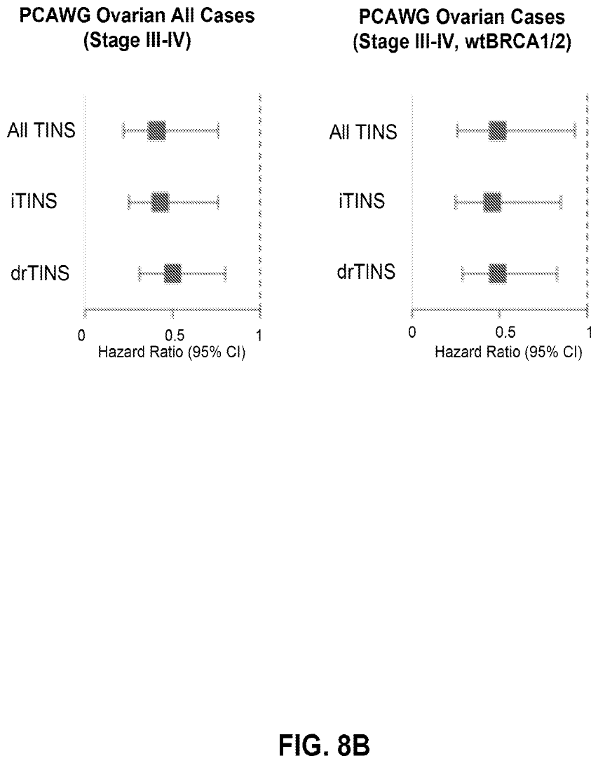

A threshold between HRD and HR competent cases were again defined by the median values of each signature for the PCAWG stage III and IV ovarian cancer cohort. HRD cases defined by all three TINS signatures were significantly associated with improved survival in the complete PCAWG stage III and IV ovarian cancer cohort, even after removing BRCA1 and BRCA2 mutated cases (FIG. 3G). Log-rank tests showed that TINS, iTINS, and drTINS are prognostic signatures for survival in ovarian cancer (FIG. 311). We noted that when using a threshold of 0, i.e., any signature measurement to define HRD cases, all three signatures remained significantly associated with survival regardless of BRCA status (FIGS. 8A, 8B).

These results demonstrate that the TINS-based screening methods disclosed herein are useful for selecting cancer patients for treatment with PARP inhibitors or platinum agents.

### Example 5: Combining TINS Signature Score with HRDetect Improves Classification of Prognostic Groups

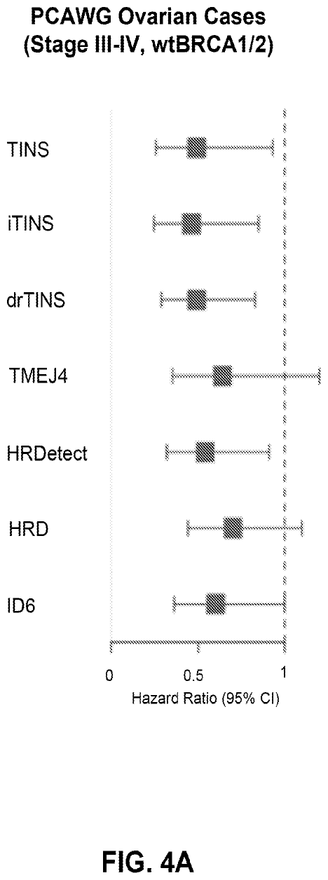

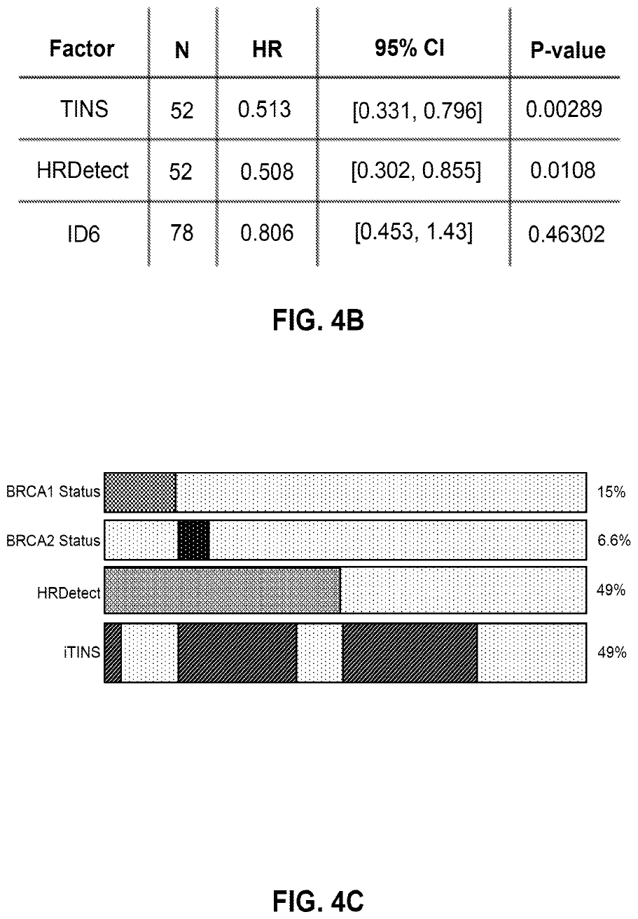

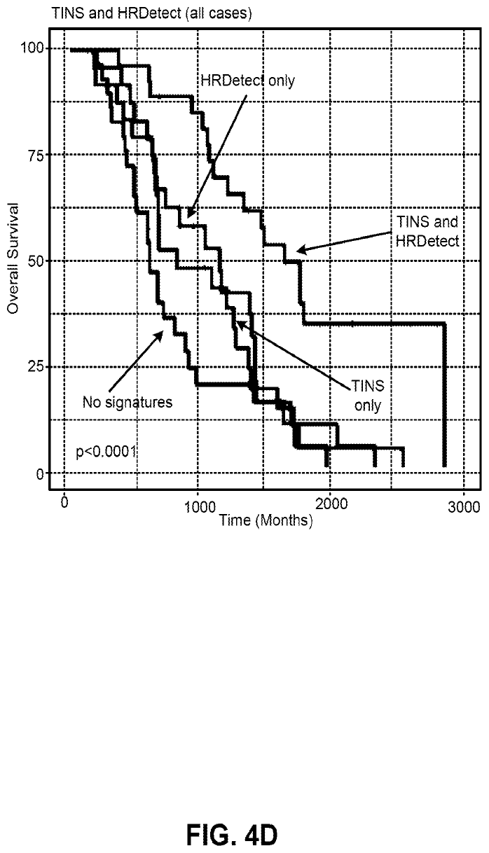

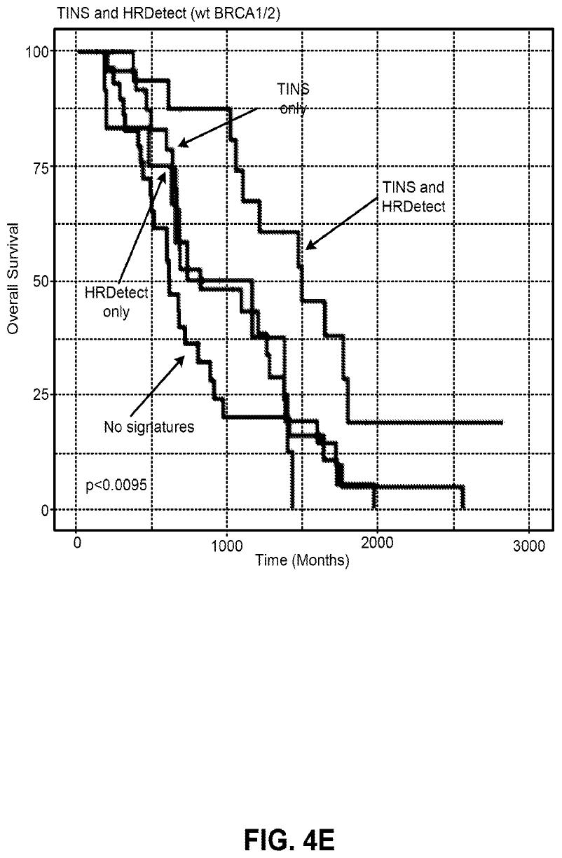

The known and novel signatures discussed can be and are used to predict patient survival in platinum-treated advanced ovarian cancer. HRD score closely resembles the Myriad MyChoice test and LOH is used by the FoundationOne test, both of which are FDA-approved. While no other signatures are presently available in a clinical setting, HRDetect, ID6, TMEJ4, and all three TINS signatures are significantly associated with survival in this patient cohort. Comparing the hazard ratios of these signatures, using TINS as the total measurement of templated insertions, revealed that only TINS, HRDetect, and ID6 are significantly associated with survival in a BRCA wild-type context (FIG. 4A). TMEJ4 performs very similarly to ID6 but does not improve the signature's ability to identify patients with HRD. Including these three signatures in a multivariate Cox regression model, TINS and HRDetect remain significantly associated with survival (FIG. 4B). In addition, TINS captured some HRD samples which HRDetect did not and vice versa, supporting the integration of both metrics (FIG. 4C).

While cases with high TINS or HRDetect are associated with improved survival individually, cases with both signatures exhibited dramatically different outcomes than those without either one, regardless of BRCA status (FIGS. 4D and 4E). Thus, when cases without known BRCA mutations are grouped into those with two signatures, median survival improves to 49.7 months compared to 20.5 months in cases without either one (p=0.0016).

One possibility is that TINS positive, HRDetect negative cohort represents cases with intermediate levels of HR capacity. The existence of a continuum of HR capacity is supported by the ARIEL3 trial, a randomization of rucaparib versus placebo in patients with relapsed high-grade serous ovarian/endometrioid/primary peritoneal/or fallopian carcinoma who had achieved at least a partial response to platinum therapy (Coleman, R. L. et al., Lancet 390, 1949-1961(2017)). PARP inhibition was associated with improved progression-free survival most clearly in the germline or somatic mutated BRCA1/2 cohort (HR 0.23). But less substantial associations were also seen in patients with high genomic LOH (HR 0.44) or even low genomic LOH (HR 0.58).

These results demonstrate that the TINS-based screening methods disclosed herein are useful for selecting cancer patients for treatment with PARP inhibitors or platinum agents.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for selecting a cancer patient for treatment with a PARP inhibitor or a platinum agent comprising:
(a) detecting levels of templated insertions (TINS) in a biological sample obtained from the cancer patient that are elevated relative to a control sample obtained from a healthy subject or a predetermined threshold; and
(b) administering to the cancer patient an effective amount of a PARP inhibitor or a platinum agent.

2. A method for prolonging survival of a cancer patient comprising
administering to the cancer patient an effective amount of a PARP inhibitor or a platinum agent,
wherein levels of templated insertions (TINS) in a biological sample obtained from the cancer patient are elevated relative to a control sample obtained from a healthy subject or a predetermined threshold.

3. The method of claim 1, wherein the levels of TINS are detected via next-generation sequencing.

4. The method of claim 1, wherein the cancer patient is diagnosed with, or is at risk of having a homologous recombination deficiency.

5. The method of claim 1, wherein the cancer patient harbors a mutation in one or more driver mutations selected from among BRCA2, BRCA1, IDH1, CDK4 and RB1.

6. The method of claim 1, wherein the cancer patient suffers from a cancer selected from among breast cancer, ovarian cancer, prostate cancer, and pancreatic cancer.

7. The method of claim 1, wherein the cancer patient exhibits stage I, stage II, stage III or stage IV cancer.

8. The method of claim 1, wherein the TINS comprise at least one of direct-repeat TINS (drTINS) and inverted TINS (iTINS).

9. The method of claim 1, wherein the TINS have a length ranging from 5 base pairs (bps) to 20 base pairs (bps) or 20 base pairs (bps) to 50 base pairs (bps).

10. The method of claim 1, wherein the PARP inhibitor is iniparib, olaparib, niraparib, rucaparib, talazoparib, veliparib, AG014699, CEP 9722, MK 4827, BMN-673, E7016, or 3-aminobenzamide.

11. The method of claim 1, wherein the platinum agent is cisplatin, carboplatin, oxaliplatin, nedaplatin, triplatin tetranitrate, phenanthriplatin, pyriplatin, picoplatin, or satraplatin.

12. The method of claim 1, wherein the biological sample obtained from the cancer patient comprises biopsied tumor tissue, whole blood, plasma, or serum.

13. The method of claim 1, further comprising determining an additional HRD signature in the cancer patient.

14. The method of claim 13, wherein the additional HRD signature comprises one or more of loss of heterozygosity (LOH), large scale state transitions (LST), and telomeric imbalance (tAI).

15. The method of claim 13, wherein the additional HRD signature comprises one or more of single base substitution signature 3 (SBS3), single base substitution signature 8 (SBS8), short tandem duplications (RefSig R3), short deletions (RefSig R5), microhomology-mediated indels, and HRD score.

16. The method of claim 13, wherein the additional HRD signature comprises ID8 signature, and ID6 signature.

17. The method of claim 1, wherein the cancer patient has not received at least one prior line of anti-cancer therapy.

18. The method of claim 1, wherein the cancer patient has received at least one prior line of anti-cancer therapy.


SEQUENCE LISTING (7 sequences)

SEQ ID NO: 1 - DNA, 137 bp, synthetic construct
  source 1..137: mol_type = other DNA
  misc_difference 1: note = a, c, t, g, unknown or other
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_difference 20: note = a, c, t, g, unknown or other
nnnnnnnnnnnnnnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 2 - DNA, 123 bp, synthetic construct
  source 1..123: mol_type = other DNA
  misc_difference 1: note = a, c, t, g, unknown or other
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_difference 20: note = a, c, t, g, unknown or other
nnnnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 3 - DNA, 110 bp, synthetic construct
  source 1..110: mol_type = other DNA
  misc_difference 1: note = a, c, t, g, unknown or other
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_difference 20: note = a, c, t, g, unknown or other
nnnnnnnnnnnnnnnnnnnngtttttgtactctcagaaatgcagaagctacaaagataaggcttcatgccgaaatcaacaccctgtcattttatggcagggtgttttttt

SEQ ID NO: 4 - DNA, 102 bp, synthetic construct
  source 1..102: mol_type = other DNA
  misc_difference 1: note = a, c, t, g, unknown or other
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_difference 20: note = a, c, t, g, unknown or other
nnnnnnnnnnnnnnnnnnnngttttagagctagaaatagcaagttaaaataaggctagtccgttatcaacttgaaaaagtggcaccgagtcggtgctttttt

SEQ ID NO: 5 - DNA, 88 bp, synthetic construct
  source 1..88: mol_type = other DNA
  misc_difference 1: note = a, c, t, g, unknown or other
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_difference 20: note = a, c, t, g, unknown or other
nnnnnnnnnnnnnnnnnnnngttttagagctagaaatagcaagttaaaataaggctagtccgttatcaacttgaaaaagtgttttttt

SEQ ID NO: 6 - DNA, 76 bp, synthetic construct
  source 1..76: mol_type = other DNA
  misc_difference 1: note = a, c, t, g, unknown or other
  misc_difference 2: note = a, c, t, g, unknown or other
  misc_difference 3: note = a, c, t, g, unknown or other
  misc_difference 4: note = a, c, t, g, unknown or other
  misc_difference 5: note = a, c, t, g, unknown or other
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
  misc_difference 11: note = a, c, t, g, unknown or other
  misc_difference 12: note = a, c, t, g, unknown or other
  misc_difference 13: note = a, c, t, g, unknown or other
  misc_difference 14: note = a, c, t, g, unknown or other
  misc_difference 15: note = a, c, t, g, unknown or other
  misc_difference 16: note = a, c, t, g, unknown or other
  misc_difference 17: note = a, c, t, g, unknown or other
  misc_difference 18: note = a, c, t, g, unknown or other
  misc_difference 19: note = a, c, t, g, unknown or other
  misc_difference 20: note = a, c, t, g, unknown or other
nnnnnnnnnnnnnnnnnnnngttttagagctagaaatagcaagttaaaataaggctagtccgttatcatttttttt

SEQ ID NO: 7 - DNA, 3978 bp, Homo sapiens
  source 1..3978: mol_type = genomic DNA
agcaatctatcagggaacggcggtggccggtgcggcgtgttcggtggcggctctggccgctcaggcgcctgcggctgggtgagcgcacgcgaggcggcgaggcggcagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcacttcatgaaattatatgaagaaaaaaccgggaacgcttggcactccaaaaatttcacgaagtatcccaaaaagttctaccccctggagattgactatggccaggatgaagaggcagtgaagaagctgacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacaatgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactgccatacgtctcagggagacccaataggcttaatcctgttgggagaagttgcccttggaaacatgtatgaactgaagcacgcttcacatatcagcaagttacccaagggcaagcacagtgtcaaaggtttgggcaaaactacccctgatccttcagctaacattagtctggatggtgtagacgttcctcttgggaccgggatttcatctggtgtgaatgacacctctctactatataacgagtacattgtctatgatattgctcaggtaaatctgaagtatctgctgaaactgaaattcaattttaagacctccctgtggtaattgggagaggtagccgagtcacacccggtggctctggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaagcttcgttagaatgtctgccttactggtttccccagggaaggaaaaatacacttccacccttttttctaagtgttcgtctttagttttgattttggaaagatgttaagcatttatttttagttaaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttgaaattcttgactttcttatgggcacttttgtcttccaaattaaaactctaccacaaatatacttacccaagggctaatagtaatactcgattaaaaatgcagatgccttctcta